(19) United States
(12) Patent Application Publication
(10) Pub. No.: US 2022/0273853 A1
(43) Pub. Date: Sep. 1, 2022

(54) Arrangement for Transporting a Liquid Through a Cannula System, Corresponding Kit and Method

(71) Applicant: ReCO2very Therapies GmbH, Bad Klosterlausnitz (DE)

(72) Inventors: Torsten HEILMANN, Bad Klosterlausnitz (DE); Sabine POST, Bad Klosterlausnitz (DE)

(21) Appl. No.: 17/637,876

(22) PCT Filed: Aug. 19, 2020
(86) PCT No.: PCT/EP2020/073245

§ 371 Date: Feb. 24, 2022

(30) Foreign Application Priority Data
Aug. 30, 2019 (EP) PCT/EP2019/073169

(51) Int. Cl. A61M 1/36; A61M 60/569; A61M 60/851; A61M 60/859; A61M 60/268

(52) U.S. Cl. CPC A61M 1/3613; A61M 60/569; A61M 60/851; A61M 60/859; A61M 1/3659; A61M 60/268; A61M 2202/0208; A61M 2210/125

(57) ABSTRACT

An arrangement (200 to 1200) is provided for transporting a liquid (B) through a cannula system (CS), comprising: a liquid guiding system (LGS) comprising at least three separated portions (SP2a, SP2b, SP3c) which define separate liquid guiding portions of the liquid guiding system (LGS), and a connecting portion (CP2 to CP12) which fluidically connects the at least three separated portions (SP2a, SP2b, SP3c) and which comprises a lumen that branches out into at least two lumens, wherein the liquid guiding system (LGS) is configured to be connected to a pump arrangement (Arr2 to An12) which drives a flow of the liquid (B), and wherein the liquid guiding system (LGS) is configured to be connected to a cannula system (CS) which is adapted to be inserted into a body of a human or of an animal and which comprises an inflow opening and an outflow opening of the liquid guiding system (LGS).

Arrangement for transporting a liquid through a cannula system, corresponding kit and method

The invention relates to liquid guiding system. Known arrangements for transporting a liquid through a cannula system may comprise a liquid guiding system, a pump arrangement comprising only one pump and a cannula system comprising two cannulas.

It is an object of the invention to provide a simple arrangement for transporting a liquid, preferably an arrangement which allows new medical applications. Furthermore, it is an object to provide a corresponding kit and corresponding methods.

This object is solved by the arrangement according to claim 1 and according to the independent claims. Embodiments are provided in the dependent claims.

The arrangement for transporting a liquid through a cannula system may comprise:

a liquid guiding system which comprises:
at least three separated portions which define separate liquid guiding portions of the liquid guiding system, and
a connecting portion which fluidically connects the at least three separated portions and which comprises a lumen that branches out into at least two lumens, e.g. there may be a bifurcation of the lumen. The lumen may be limited circumferentially by a wall of the connecting portion. The connecting portion may comprise at least three ports which are connected by the lumen.

Examples for connecting portions are:

T-connectors having three ports wherein two ports and their connection may be arranged on a first straight line and one port at a line which is perpendicular to the first straight line, or

Y-connector having three ports, or

X-Connectors having four ports, or

integral connecting portions comprising tubes which are molded to the connecting portions.

Y-connectors may be preferred because of better fluid flow characteristics compared with other types of connectors. A symmetric Y-connector may be used, comprising for instance three separated portions, wherein each pair of adjacent separated portion includes an angle of 120 degrees. Alternatively, the angle between two of the separated portions may be less than the other angles. The angle between two of the separation portions may be in the range of 100 degrees to 50 degrees or more preferred within the range of 80 degrees to 50 degrees. The common portion that is mentioned in this application may include an angle of more than 120 degrees or more than 130 degrees to the two other separated portions respectively, especially the same angle. All three angles may sum up to 360 degrees.

The liquid guiding system may be configured to be or may be connected to a pump arrangement which drives a flow of the liquid. The pump arrangement may be part of the arrangement for transporting a liquid or it may be not be part of the arrangement for transporting a liquid.

The liquid guiding system may be configured to be or may be connected to a cannula system which is adapted to be inserted into a body of a human or of an animal and which comprises an inflow opening and an outflow opening of the liquid guiding system. The cannula system may be part of the arrangement for transporting a liquid or it may be not be part of the arrangement for transporting a liquid. The cannula system may comprise at least one cannula.

The liquid may be blood, saline, or another medical liquid.

The connecting portion comprising a branching lumen opens many possibilities for the realization of medical applications. It is for instance possible to connect at least two pump devices in parallel by using the connecting portion. It is possible to connect at least two pump devices in series if for instance two connecting portion are used.

However, alternatively it is possible to use only one pump device. It is for instance possible to use valves within the liquid guiding system in order to define flow directions through the connecting portion. It is possible to include a pump which pumps blood bi-directionally, for instance into one direction and thereafter in the opposite direction, preferably periodically. Using the valves and the connecting portion may allow nevertheless providing a circulation.

Known pump devices, especially already certified pump devices, may be used therefor for known application using higher pumping power and/or higher pumping volume and/or for new medical applications. Examples for pump devices are membrane pump devices, peristaltic (roller) pumping devices, centrifugal pump devices, axial pump devices, diagonal pump devices, etc. Shear stress to the liquid should be as low as possible, especially if the liquid is blood. Shear stress may destroy particles within the liquid, especially particles/molecules which are biological active and/or which have to fulfill a complex function. Furthermore, shear stress may promote clotting of the liquid, especially of blood. Blood clotting may be detrimental for the health of a patient which is treated using the arrangement for transporting the liquid. Membrane pumps may have a low shear stress impact onto the liquid.

A first separated portion of the at least three separated portions and a second separated portion of the at least three separated portions may be configured to be or may be connected to a pump arrangement which drives a flow of the liquid through the liquid guiding system. The first separated portion may be different from the second separated portion. The arrangement may be configured such that a third separated portion of the at least three separated portions may be a common inlet and outlet portion through which flow flows coming from the first separated portion and from the second separated portion and/or through which third separated portion flow flows which then flows to the first separated portion and to the second separated portion. Thus, it is possible to operate two pump devices or more than two pump devices in parallel, e.g. the flow may be added allowing to drive the liquid with a higher volume per minute. If for instance two 40 ml (milliliter) membrane pumps are connected in parallel a pumping volume of 80 ml results. If the pump pumps once every second the resulting pumping volume per minute is 4.81 (liter) per minute which is quite good if compared with the pumping volume of a healthy heart of for instance 5 1/min.

The arrangement may comprise the pump arrangement. The pump arrangement may comprise at least two pump devices. A first pump device of the at least two pump devices may be configured to be or may be connected to the first separated portion. A second pump device of the at least two pump devices may be configured to be or is connected to the second separated portion. The connecting portion distributes fluid in a fluid flow which is directed to the first separated portion and to the second separated portion to these portions. Fluid flow from the first separated portion and form the second separated portion is collected or united into a common fluid flow by the connecting portion.

Each of the at least three separated portions may be configured to allow a bidirectional flow of the liquid. This may allow using membrane pumps which are operated in parallel. Other pumps may be used as well, for instance in combination with a variable volume reservoir, e.g. a balloon or a piston arrangement.

Preferably, no one-way valves may be used within the arrangement for transporting the liquid. The pump arrangement may be free of a one-way valve. Thus, there may be a simple construction of the arrangement for transporting the liquid and especially of the pump arrangement which includes at least two pump devices or only one pump device. Furthermore, tubes without valves may result in less clotting of the liquid, especially of blood, compared to tubes which comprise valves.

Other names for a one-way valve are “check valve” or “non-return” valve. The one-way valves may be arranged within at least two separated portions and/or within a connecting portion.

However, there may be an internal valve or an internal valve function within a cannula to which the arrangement is connected or which is part of the arrangement, e.g. within a bidirectional cannula. The cannula may be configured to be inserted into the body, e.g. with at least 50 percent or at least 75 percent of its length. Thus, the cannula may be or is different from tubes of the arrangement which tubes are usually not intended to be inserted into a body of a patient.

A bidirectional cannula may be configured to be or may be connected to the third separated portion which may be a common portion to the first separated portion and to the second separated portion because flow to these two portion or flow coming from these portions flows also through the common portion.

The arrangement may comprise the cannula system. The cannula system may comprise:

two single lumen cannulas, preferably configured for right ventricle assist (pRVAD) or for left ventricle assist (pLVAD®) or for bi ventricle assist (pBiVAD®) or for pulmonary artery to left atrium or left ventricle blood transport or for veno-arterial extracorporeal membrane oxygenation, or

a dual lumen cannula system comprising at least one inner cannula which is inserted into an outer cannula, preferably configured for left ventricle assist (pLVAD) or for biventricular assist (pBiVAD®), or

a bidirectional cannula which is configured to be connected to the third separated portion, preferably configured for renal assist or for right ventricle assist (pRVAD®) or for pulmonary artery to left atrium or left ventricle blood transport.

The cannula(s) may be introduced jugular. This may allow usage of cannulas with greater outer diameter compared with femoral access. Furthermore, a cannula which is inserted jugular into the heart may be shorter than a cannula which is inserted femoral. Both aspects may have an influence to the pump, e.g. higher pumping volume may be possible etc.

The bidirectional cannula may have a length in the range of 40 cm (centimeters) to 50 cm, e.g. the length may be less than 80 cm and more preferably less than 60 cm. The outer diameter of the cannula(s) may be in the range of 19 Fr (French, 1 French equal to 0.33 mm (millimeter) or 1/3 mm) to 31 Fr, e.g. within the range of 21 Fr to 29 Fr. Higher flows, e.g. flows per minute, may be possible. This applies especially to the renal medical applications.

Alternatively, the arrangement may have the following features:

the first separated portion of the at least three separated portions may be an inflow portion of the liquid guiding system, and/or

the second separated portion of the at least three separated portions is an outflow portion of the liquid guiding system, and/or

the first separated portion may be different from the second separated portion, and/or

the third separated portion of the at least three separated portions may be configured to be or may be connected to a pump arrangement which drives a flow of the liquid.

The arrangement may be configured such that the third separated portion is a portion through which flow coming from the first separated portion flows and/or through which flow flows which flows then to the second separated portion.

This alternative arrangement may allow other medical applications, e.g. if further valves are used within the arrangement for transporting the liquid. Furthermore, it may be possible to realize pump arrangements which have special technical effects and advantages, for instance realizing pulsatile liquid flows, preferably with at least one oxygenator device.

The first separated portion or only the first separated portion but not the second separated portion may comprise a one-way valve. One-way valves of Quest Medical (may be a trademark) may be used or of other manufacturers. Alternatively, the second separated portion or only the second separated portion but not the first separated portion may comprises a one-way valve. Further, alternatively, the first separated portion may comprise a first one-way valve and the second separated portion may comprise a second one-way valve. An oxygenator device or another device for treating the liquid, especially blood, may have an inherent valve function with creates a directional flow. The usage of one-way valves may be a simple solution to generate directed flows. One-way valves within the first separated portion and within the second separated portion may allow the usage of bidirectional pumps arrangements, e.g. of membrane pumps.

The arrangement which comprises at least one one-way valve may comprise the pump arrangement. The pump arrangement may comprise only one pump device. The pump device may be configured to be or may be connected to the third separated portion. If only one pump device is used the arrangement may have as few parts as possible. Only one pump device may be sufficiently for some medical applications, preferably in combination with using valves, e.g. one-way valves.

The arrangement which comprises only one pump device and for instance at least one one-way valve may comprise the cannula system. The cannula system may comprise:

two single lumen cannulas defining an inflow opening and an outflow opening of the liquid guiding system. The single lumen cannula may not be arranged within another cannula and no other cannula arranged within cannula. The two single lumen cannulas may preferably be configured for left ventricle assist (pLVAD®) or for pulmonary artery to left atrium or left ventricle blood transport.

a dual lumen cannula system which comprises at least one inner cannula which is arranged inside of an outer cannula. Especially, dual lumen cannula system which allow separate insertion of both cannulas into the body may be advantageous, e.g. with regard to reducing trauma of blood vessels. The dual lumen cannula system may be preferably configured for left ventricle assist (pLVAD®).

a bidirectional cannula defining an inflow opening and an outflow opening of the liquid guiding system and comprising at least one internal valve or internal valve function. Although the bidirectional cannula comprise an internal valve or an internal valve function and although only one pump device is used it is possible to establish an outer flow circuit with only one direction. This may allow the usage of liquid treatment devices which may be operated only with a flow which is directed in one direction, e.g. some kinds of blood filters, some kinds of oxygenator devices, etc. The bidirectional cannula may preferably configured for pulmonary artery to left atrium or left ventricle blood transport

The arrangement comprising only one pump device may comprise a device for carbon dioxide removal.

The arrangement may be configured to be or may be connected with the device for carbon dioxide removal from the liquid but not with an oxygenator device which enhances oxygen in the liquid. The pumping power or pump volume may be sufficiently for an extracorporeal carbon dioxide removal ECCO2R. Contrary, oxygenator devices may need a higher pumping power than compared with the pumping power of the used pumping device.

Alternatively, the arrangement with the third separated portion connected to the pump device and with optional valves may also comprise the pump arrangement. The pump arrangement may comprise at least two pump devices, preferably two pump devices. This may allow a multiplication of the pump volume and therewith of the pump volume per minute. There may be pump devices in the marked that are certified and of a type that is used already a long time because the pump devices are very reliable.However, only the usage of two of these pumping devices may allow special medical applications. A further technical effect is that one of the pumps may be replaced during operation of the arrangement for liquid transport by the other of the pumps. Changing of pumps may be easier if further valves and cocks (shut-off valve, stop valve) are used, e.g. a three way stop cock.

The pumps may be operated in series, e.g. the liquid flow goes through the pump devices in a serial manner, e.g. one after the other. Alternatively, the pump devices may be connected in parallel, e.g. the fluid flow sums up and a part of the fluid flow goes to one pump device and another part of the fluid flow goes through the other pump device. Furthermore, combinations of serial pump devices and of parallel pump devices are possible as well.

The first pump device and the second pump device may be different pump devices or may at least have some parts which are different from each other and some parts in common, for instance if only one intra-aortic balloon pump IABP console/device is used or if other devices are used which enable a synchronous operation of the pump devices.

Both pump devices may be configured to be or may be fluidically connected in parallel. If parallel operation is used, the arrangement may comprise the cannula system. The cannula system may comprise cannulas having a lager diameter compared to cannula systems which are connected to only one pump:

two single lumen cannulas defining an inflow opening and an outflow opening of the liquid guiding system. A percutaneous left ventricle assisted device pLVAD® may be realized using two single lumen cannulas, which are preferably both inserted through the septum, e.g. the atrial septum of the heart. Alternatively, a drainage from the pulmonary artery to left atrium and/or to left ventricle may be realized using two single lumen cannulas.

a dual lumen cannula system which comprises at least one inner cannula which is arranged inside of an outer cannula. A percutaneous left ventricle assisted device pLVAD® may be realized using a dual lumen cannula system, which is inserted transseptal. Furthermore, bi-ventricle assisted devices pBiVAD® may be realized as is described below in more detail.

a bidirectional cannula defining an inflow opening and an outflow opening of the liquid guiding system and comprising at least one internal valve or internal valve function. A drainage from the pulmonary artery to left atrium and/or to left ventricle may be realized using a bidirectional cannula.

The arrangement which comprises two pump devices in parallel may be configured to be or may be connected to an oxygenator device which enhances oxygen in the liquid. Oxygenator devices may need comparably high flow volumes per minute in order to deliver the complete oxygen or an essential part of the oxygen for a patient.

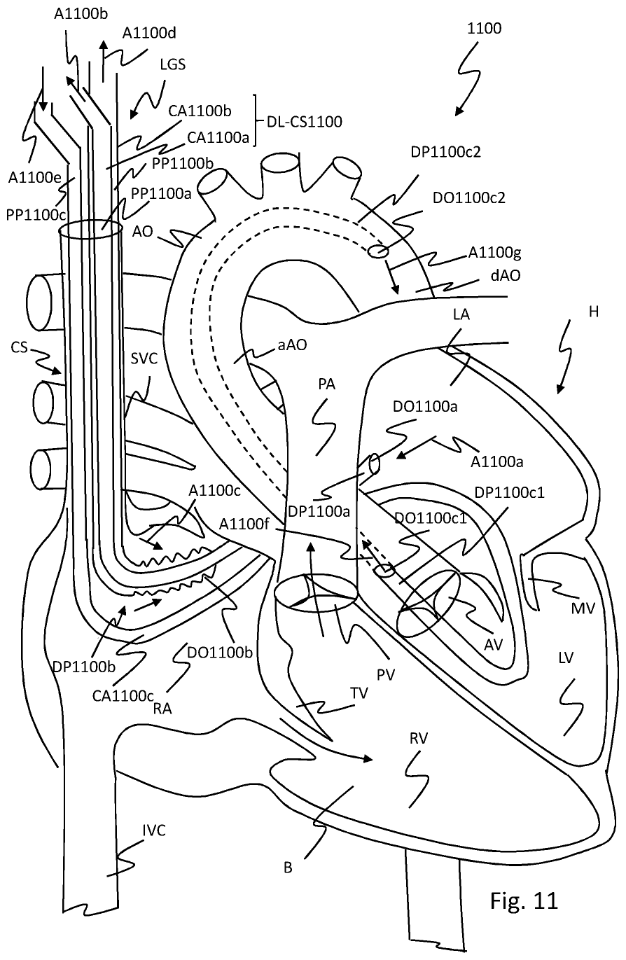

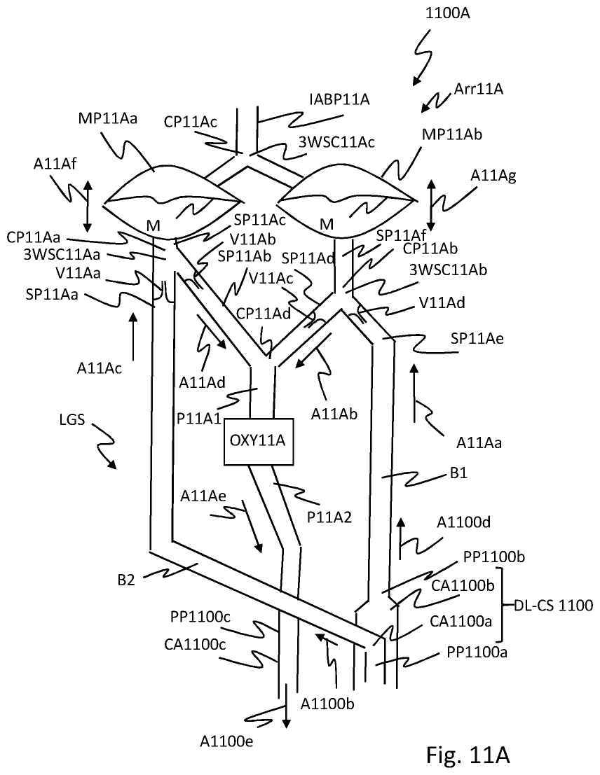

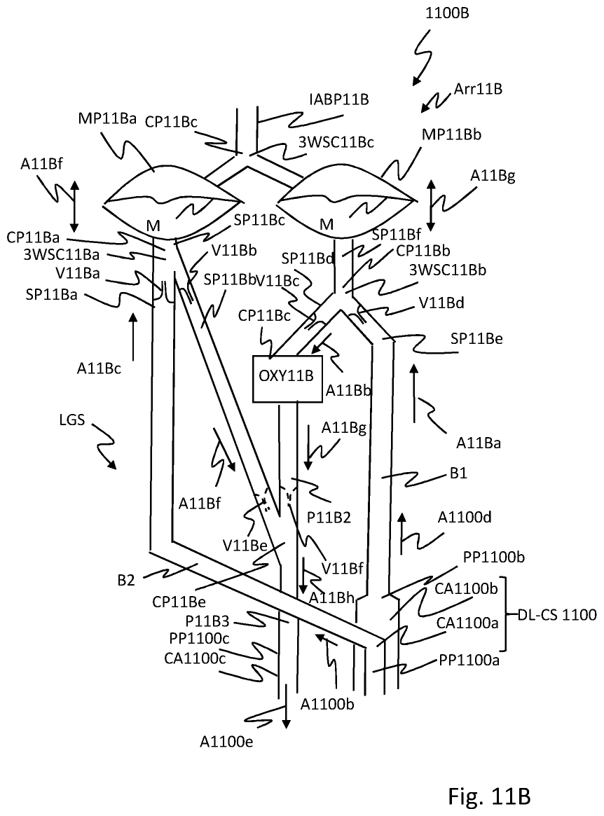

The arrangement comprising two pump devices which are connected in parallel may comprise an oxygenator device which enhances oxygen in the liquid, see for instance FIG. 11A. The arrangement may be configured to be or may be connected to the oxygenator device which enhances oxygen in the liquid.

The arrangement may be configured such that the outflow of both pump devices flows through the oxygenator device, preferably before entering the outflow cannula again. This may allow high amounts of oxygen to be introduced into the liquid, for instance into blood. Preferably, the arrangement may comprise the cannula system. The cannula system may be configured to be or may be connected to a dual lumen cannula system which comprises at least one inner cannula which is arranged inside of an outer cannula.

Furthermore, the cannula system may comprise a further single lumen cannula. Thus, a biventricular assisted device (pBiVAD) is provided. The dual lumen cannula system may drain or suck blood from the left atrium and from the right atrium. The third single lumen cannula may be used to deliver blood back into the heart, for instance into the left ventricle, into ascending aorta or into descending aorta. All cannulas may be inserted through the atrial septum.

In an alternative (see FIG. 11B), the arrangement comprising two pump devices which are connected in parallel may also comprise an oxygenator device which enhances oxygen in the liquid. The arrangement may be configured to be or may be connected to the oxygenator device. The arrangement may be configured such that the outflow of one pump device, preferably of only one pump device, of at least two pump devices flows through the oxygenator device but not the outflow of the other pump device of the at least two pump devices, preferably before entering an outflow cannula again. Thus it may not be necessary to enhance some of the drained blood, especially blood which was already enhanced with oxygen by the lung of a patient. Furthermore, this variant makes it possible that a pulsatile outflow is generated which is more natural and therefore more advantageous compared to a continuous blood flow, for instance with regard to the prevention of blood clotting. Thus, the pumping power/volume of two pumps in parallel may be used and a pulsatile blood flow. Preferably the arrangement may comprise the cannula system and may be configured to be or is connected to a dual lumen cannula system which comprises at least one inner cannula which is arranged inside of an outer cannula and to a single lumen cannula. Furthermore, the cannula system may comprise a further single lumen cannula. Thus, a biventricular assisted device (pBiVAD®) is provided. The dual lumen cannula system may drain or suck blood from the left atrium and from the right atrium. The third single lumen cannula may be used to deliver blood back into the heart, for instance into the left ventricle, into ascending aorta or into descending aorta. All cannulas may be inserted through the atrial septum. See for instance FIG. 11B for an example.

Alternatively, within the arrangement which comprises several pump devices the at least two pump devices may be configured to be or may be connected fluidically in series. Also in this case, the arrangement may comprise the cannula system. The cannula system may comprise:

two single lumen cannulas defining an inflow opening and an outflow opening of the liquid guiding system. A percutaneous right ventricle assisted device (pRVAD®) may be realized. Furthermore, a percutaneous biventricular assisted device (pBiVAD®) may be realized, preferably if a single lumen cannula is used which allows blood drainage from the left atrium and from the right atrium, e.g. by inserting the cannula transseptally and by using two groups of holes, wherein each group comprises at least one hole or opening. Moreover, a veno-arterial extracorporeal membrane oxygenation (ECMO) may be realized using two single lumen cannulas.

a dual lumen cannula system which comprises at least one inner cannula which is arranged inside of an outer cannula, or

a bidirectional cannula which comprises an inflow opening and an outflow opening of the liquid guiding system and comprising at least one internal valve or internal valve function. A percutaneous right ventricle assisted device (pRVAD) may be realized.

The arrangement comprising at least two pump devices in series may comprise an oxygenator device. The arrangement is configured to be or is connected to an oxygenator device which enhances oxygen in the liquid. Thus, it may be possible to push and to pill liquid through the oxygenator device. Furthermore, it may be possible to provide a pulsatile outflow of the arrangement into the body of a patient.

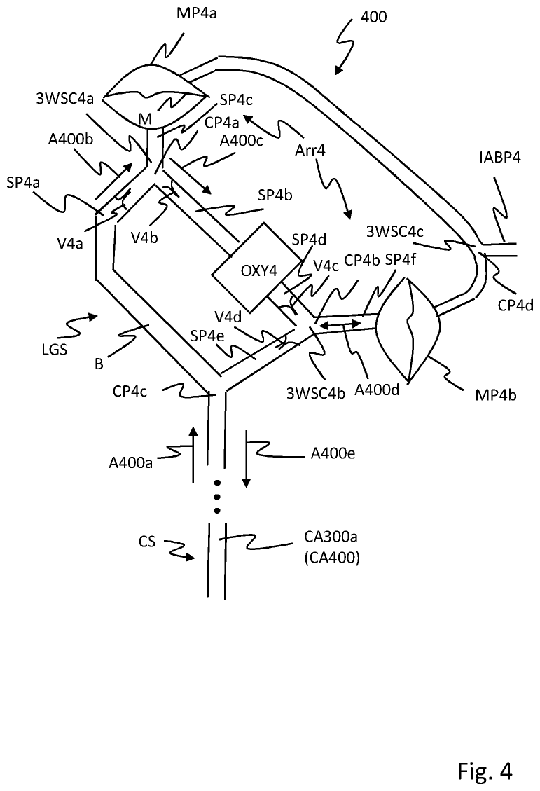

The arrangement may be configured such that the outflow of only one pump device or of only some of the pump devices of the at least two pump devices flows through the oxygenator device and that the outflow of the oxygenator device flows through at least one other pump device of the at least two pump devices. Examples are mentioned below in connection with FIGS. 4, 5, 8 and 12. This arrangement enables pulsatile outflow and complete oxygenation of the blood. Furthermore, this arrangement is simple and/or comprises less tubes than other arrangement comprising two pump devices and an oxygenator device.

All arrangements mentioned above may comprise the pump arrangement. The pump arrangement may comprise at least one pump device for driving the liquid through the liquid guiding system. The arrangement may be configured to be connected or may be connected to the at least one pump device. The at least one pump device may comprise one port, preferably only one port, through which the liquid is transported in two opposite directions, e.g. bidirectionally and/or periodically. This kind of liquid transport, especially of blood transport may reduce blood clotting considerably.

The at least one port may be inlet and the outlet (inflow and outflow) of a variable volume reservoir. The port may comprise only one lumen. The variable volume reservoir may be realized by a plunger arrangement. Preferably, the variable volume reservoir is part of a membrane pump. An inflatable balloon or an inflatable torus or an inflatable flat membrane may be used within the variable volume reservoir.

The at least one pump device may be a membrane pump which comprises at least one flexible membrane. The membrane may separate a case of the membrane pump in a variable volume reservoir chamber and in a compensation chamber and/or driving chamber. The membrane may be a torus membrane or a “flat” and/or sheet membrane.

The arrangement may be configured such that the membrane pump device may be driven by an intra-aortic balloon pump device/console.

At least two membrane pump devices of the pump arrangement may be driven by the same intra-aortic balloon pump console. These, IABP console are widely provided in many hospitals. The IABP console may comprise a control unit which controls the gas inflow and outflow of the console (for instance helium or air) depending on electrode signals of a heart within the body. If only one console is used to drive two membrane pump devices (variable volume reservoirs) both membrane pump devices operate synchronously. Change of one pump device during operation of the other pump device may be possible. Certified pumps, especially membrane pumps may be used, e.g. for instance 40 ml membrane pumps.

Pumping may be performed each heartbeat, each second heartbeat, or in another appropriate manner. It was surprisingly that the known IABPB consoles are able to operate for instance two 40 ml membrane pumps, e.g. a volume greater as 60 ml, greater as 70 ml or grater as 80 ml but for instance smaller than 200 ml.

A three-port connector may be used to distribute a gaseous fluid coming from the intra-aortic balloon pump console or device to the at least two membrane pump devices of the pump arrangement. If more than two membrane pumps are operated at the same IABP console a multi-port connector comprising more than three ports may be used.

The arrangement may comprise at least two three way stop cocks which may be configured to enable changing and/or removal and/or stopping of at least one pump device of the pump arrangement during continuous operation of the arrangement by at least one other pump device of the pump arrangement. Changing pumps during operation may mean without interruption of the flow of the liquid. Thus, life supporting blood flow may be provided via several pump changing phases, for instance for a time greater than three hours, greater than 20 hours or even greater than days or weeks, for instance greater than at least one day or greater than at least one week. At least 3, 5 or 10 changes may be made without interruption of a blood flow. Changing of pump devices may prevent clotting of blood in the pumps. The used pump devices may be cleaned and used again. Removal or stopping of one pump device may be relevant for weaning, i.e. if the heart takes over its full function again.

The cannula system may comprise a single lumen cannula which may be a unidirectional cannula and which comprises at least one backflow prevention valve, preferably a one-way valve. The unidirectional cannula may have a proximal end and a distal end. There may be a unidirectional flow within the whole lumen between the proximal end and a distal end. Alternatively and/or additionally, the cannula system may comprise a dual lumen cannula system comprising an outer cannula and an inner cannula which may be inserted into the outer cannula preferably within the body. The inner cannula and/or the outer cannula may be a unidirectional cannula comprising at least one backflow prevention valve, preferably a one-way valve. The backflow prevention valve may be arranged at or within a distal portion of the cannula. These further one-way valves may prevent that blood flows into outflow openings which are mainly used as outflow openings or that blood flows out of inflow openings which are mainly used as inflow openings.

Alternatively and/or additionally, there may be one-way valves or other valves having the function of backflow prevention valves within an intermediated portions of the cannulas which have the same purpose. This is similar to flaps within the veins of the human body which flaps prevent backflow during the systole.

The backflow prevention valves may be used independently of the outer connection of the respective cannula, preferably independent of the pump arrangement that is used and/or independent of the medical application.

The arrangement which comprises at least two pump devices may comprise at least four or at least five multi-port elements each comprising at least three ports and at least four one-way valves. The multi-port elements may be Y-Connectors, T-connectors, etc. Alternatively, the multi-port elements may be integral branches of a liquid guiding system. Complete decoupling of at least two pumps from each other may be possible although the pumps are connected fluidly in parallel.

There may be the following circuit:

A first multi-port element comprises a common port which is connected with a single fluid port of a first membrane pump, an inlet port and an outlet port.

A second multi-port element comprises a common port which is connected with a single fluid port of a second membrane pump, an inlet port and an outlet port.

A third multi-port element comprises a first inlet port, a second inlet port and a common outlet port, wherein the first inlet port of the third multi-port element is connected to the outlet port of the first multi-port element, and wherein the second inlet port of the third multi-port element is connected to the outlet port of the second multi-port element.

A fourth multi-port element comprises a common inlet port, a first outlet port and a second outlet port, wherein the first outlet port of the fourth multi-port element is connected to the inlet port of the first multi-port element, and wherein the second outlet port of the fourth multi-port element is connected to the inlet port of the second multi-port element, and

Preferably an optional fifth multi-port element comprises a common inlet port and outlet port, an outlet port and an inlet port,

wherein the outlet port of the fifth multi-port element is connected to the common inlet port of the fourth multi-port element, and wherein the inlet port of the fifth multi-port element is connected to the common outlet port of the third multi-port element.

The circuit may be simple and easy to establish if Y-connectors are used as multi-port elements:

A first Y-connector comprises a common port which is connected with a single fluid port of a first membrane pump, an inlet port and an outlet port.

A second Y-connector comprises a common port which is connected with a single fluid port of a second membrane pump, an inlet port and an outlet port.

A third Y-connector comprises a first inlet port, a second inlet port and a common outlet port, wherein the first inlet port of the third Y-connector is connected to the outlet port of the first Y-connector, and wherein the second inlet port of the third Y-connector is connected to the outlet port of the second Y-connector.

A fourth Y-connector comprises a common inlet port, a first outlet port and a second outlet port, wherein the first outlet port of the fourth Y-connector is connected to the inlet port of the first Y-connector, and wherein the second outlet port of the fourth Y-connector is connected to the inlet port of the second Y-connector.

Preferably a fifth Y-connector comprises a common inlet port and outlet port, an outlet port and an inlet port,

wherein the outlet port of the fifth Y-connector is connected to the common inlet port of the fourth Y-connector, and wherein the inlet port of the fifth Y-connector is connected to the common outlet port of the third Y-connector.

The arrangement may comprise:

a variable volume reservoir comprising:
a case, a membrane within the case, wherein the membrane separates a reservoir chamber within the case from a compensation chamber within the case, and a reservoir port that is connected to the reservoir chamber,

a multi-port element which comprises the at least three separated portions and a connecting portion, wherein the connecting portion comprises a lumen that branches out into at least two lumens,

an input flow connection which is configured to be or which is fluidically connected with a first separated portion of the at least three separated portions,

an output flow connection which is configured to be or which is fluidically connected with a second separated portion of the at least three separated portions,

wherein the reservoir port is configured to be or which is fluidically connected with a third portion of the at least three separated portions,

an input one-way valve within the first separated portion or within the input flow connection, wherein the input one-way valve allows flow in an input flow direction which is directed from the first separated portion to the third separated portion and which blocks flow in the opposite direction, e.g. from the third separated portion to the first separated portion, and/or

an output one-way valve within the second separated portion or within the output flow connection, wherein the output one-way valve allows flow in an output flow direction which is directed from the third separated portion to the second separated portion and which blocks flow in the opposite direction, e.g. from the second separated portion to the third separated portion.

The input flow connection may be a connection from a cannula to the first separated portion. The output flow connection may be a connection from second separated portion to the same cannula (e.g. bidirectional cannula) or to another cannula. Shear stress may be low in the arrangement, preferably because a membrane is used, e.g. there may be no rotating parts which may destroy blood particles. Destroyed blood particles may enhance blood clotting. This means that there may be less blood clotting if less blood particles are destroyed.

Furthermore, a kit is provided which comprises the elements of the arrangement according to any one of the embodiments mentioned above, for instance within a bag, cartoon, box etc. There may be a manual included which describes the arrangement in detail. Some parts of the arrangement may already be assembled in the kit. However other parts may be assembled by a physician, for instance after a cannula has been inserted.

Furthermore a method of using the arrangement is provided wherein at least one intra-aortic balloon pump console is used to drive the liquid flow. At least two pump devices of the pump arrangement may be driven by the same the same intra-aortic balloon pump console. The technical effect(s) is/are mentioned above.

Furthermore, a method of using the arrangement according to any one of the embodiments mentioned above for any one of the following medical applications is provided:

renal support/assist, preferably using bidirectional cannula,

right ventricle support/assist (pRVAD®), preferably using a bidirectional cannula which is inserted into an outer cannula or two single lumen cannulas,

left ventricle support/assist (pLVAD®), preferably using two single lumen cannulas or a dual lumen cannula system,

bi-ventricle support/assist (pBiVAD®), preferably using two single lumen cannulas or alternatively a dual lumen cannula system and a single lumen cannula with extra corporeal membrane oxygenation (ECMO) or without extra corporeal membrane oxygenation ECMO,

pulmonary artery drain to left atrium or to left ventricle or to aorta, preferably with carbon dioxide removal, preferably using a bidirectional cannula which is inserted into an outer cannula or using two single lumen cannulas,

veno-arterial extra corporeal membrane oxygenation (V-A ECMO), preferably using two single lumen cannulas.

Other medical application are possible as well.

A bi-ventricle support/assist (pBiVAD R) may be realized using a single lumen cannula which is configured to drain blood from the left atrium through at least one first opening and to drain blood from the right atrium through at least one second opening. Alternatively, a biventricular support/assist (pBiVAD®) may be realized using a dual lumen cannula system which may be configured to drain blood from the left atrium through at least one first opening of a first cannula of the dual lumen cannula system and to drain blood from the right atrium through at least one second opening of a second cannula of the dual lumen cannula system. The first cannula may be is inserted into the second cannula within the body of a patient in order to reduce trauma or damage to vessels during insertion of the second cannula. In both cases at least one of the cannulas may be inserted transseptal, e.g. through the atrial septum. In both cases, variable diameter arrangements, e.g. cage arrangements and/or balloons may be used, around at least one of the first hole/opening or second hole/opening. Membranes may also be used on the cage arrangement(s).

In all cases that mention cannulas in this description, the cannula(s) may have at least one variable diameter arrangement, e.g. a cage arrangements and/or balloon. The variable diameter arrangement may be located around at least one hole/opening of the cannula. At least one membranes may also be used on the cage arrangement(s).

The basic principle of an endovascular catheter/cannula therapy may be a treatment of vessels and/or by using vessels for the advancement of a catheter, for instance plastic tubes or plastic tubes that are armed with metal. An incision may be made into the skin of a patient. The incision may have a length that is less than 5 cm (centimeter), less than 3 cm or less than 1 cm. Local anesthesia may be used thereby. An auxiliary cannula may be used to insert a guide wire and/or dilators to expand the incision and/or an opening within the vessel. The catheter or cannula may then be inserted using the guide wire and/or an introducing member.

No thoracotomy may be necessary if cannulas or catheters are used. A cannula may be a tube that can be inserted into the body, often for the delivery or removal of fluid or for the gathering of data. A catheter may be a thin tube made from medical grade materials serving a broad range of functions. Catheters may be medical devices that can be inserted into the body to treat diseases or to perform a surgical procedure.

Both terms “cannula” and “catheter” are used interchangeably in the following if not stated otherwise. No special surgery may be necessary, i.e. it may not be necessary that a very high specialized physician or surgeon uses the proposed cannula and/or performs the proposed methods.

By modifying the material or adjusting the way cannulas or catheters are manufactured, it is possible to tailor them for cardiovascular, urological, gastrointestinal, neurovascular, and ophthalmic applications. A catheter or cannula may be left inside the body, either temporarily or permanently. A permanently catheter or cannula may be referred to as an “indwelling catheter or cannula” (for example, a peripherally inserted central catheter or cannula).

Catheters and cannulas may be inserted into a body cavity, duct or vessel. Functionally, they allow delivery and/or drainage of fluids, administration of fluids or gases, access by surgical instruments, and/or also perform a wide variety of other tasks depending on the type of catheter or cannula. The process of inserting a catheter is “catheterization”. The process of inserting a cannula is “cannulization”. In most uses, a catheter or cannula is a thin, flexible tube (“soft”) though catheters or cannulas are available in varying levels of stiffness depending on the application.

The variable volume reservoir may comprise a casing and a flexible membrane within the casing. Alternatively other types of membrane pumps, a piston arrangement, a bellow etc. may be used. The advantage may be that there may be no rotating parts that are in contact with the blood of a subject. No shear stress or only low shear stress may be impacted to blood molecules. This may result in no damage or only less damage of blood molecules. Thus these molecules may fulfill their complex natural function further, e.g. oxygen transport, immune functions, etc.

The variable volume reservoir may comprise at least one membrane, preferably a flat membrane or a toroidal membrane. The membrane may be made of polycarbonate, poly(methyl methacrylate) PMMA, silicone, or of another appropriate material.

The usage of a membrane opens the possibility for a good temperature control of the blood.

The inflation/deflation frequency may be in the range of 60 to 90 times per minute or in the range of 70 to 80 times per minute. Thus, every heart beat may be used to deliver blood into the blood circuit of subject. Alternatively, it may be advantageously to deliver blood only every second heartbeat or every third heartbeat for instance in order to improve timing based for instance on ECG (electrocardiography) data or signals or on other signal. A timing rate of 50/50 may be used for pumping blood into the body and out of the body. However, other timing rates are also possible, for instance more time for pumping blood into the body and less time for pumping blood out of the body or vice versa. The difference may be at least 10 percent or twenty percent of the greater value. There may be no pause between the switching of the direction of blood flow in the proximal part of the cannula, i.e. no pause that is longer as a minimum that is technically necessary for switching. Alternatively, there may be a pause or a longer pause between the switching from drainage to delivery and vice versa.

The displacement device, e.g. variable volume reservoir, or the pump may be arranged near the body of a subject to allow short fluidic circuitries. The distance between the entry point of the cannula into the body and the variable volume reservoir/pump may be in the range of 5 cm (centimeter) to 15 cm and may be less than for instance 20 cm.

The variable volume reservoir may be adapted to be used with an IABP (Intra-Aortic Balloon Pump) console that is not part of the assembly. Alternatively, the assembly may comprise a control unit that is able to control the variable volume reservoir or the pump depending on the heartbeat and/or on pulse beat that is measured by at least one sensor, for instance a known IABP (Intra-Aortic Balloon Pump) or another control unit. IABP (Intra-Aortic Balloon Pump) devices are widely used for other purposes in many clinics and hospitals. Thus, there may be no extra costs involved for these control units. The control unit may receive ECG (electrocardiography) signal or other signals or data that allow control of the variable volume reservoir or of an equivalent pump.

The variable volume reservoir may have a maximal overall pump volume that is equal to or greater than 50 ml (milliliter) or equal to or greater of 60 ml, preferably within the range of 60 ml to 160 ml or most preferably within the range of 80 ml to 120 ml. This volume may refer to the difference of the volumes between the expulsion phase and the aspiration phase. A higher volume may allow a higher pumping rate.

The volume may be appropriately selected with regard to the volume of the lumen portion of the cannula/catheter and/or a conduit between the cannula and an input port of the variable volume reservoir. Preferably, the variable volume of the variable volume reservoir may be greater than the sum of the volume of the lumen portion of the cannula and the volume of the conduit. The variable volume of the variable volume reservoir may be for instance within the range of plus 5 percent to 30 percent of the sum of the volumes of the lumen portion and of the conduit. This may result in low or no blood clotting. Good oxygenation may be reached if an oxygenator is used. No dead ends may be generated within the circuitry if both volumes are selected appropriately.

In this application document the definition for “distal” is far from a person that inserts the cannula or catheter. “Proximal” means near to the person that inserts the cannula or catheter. In the following the longitudinal axis of the lumen portion or the extension thereof beyond the lumen portion may be used as a reference axis. The terms “radial”, “axial” and/or “angularly” may be used with regard to this reference axis. This may be similar to the usage of cylinder coordinates that are used in a cylindrical coordinate system.

The proposed method and its embodiments may not be used for treatment of the human or animal body by surgery or therapy and may not be a diagnostic method practiced on the human or animal body. Alternatively, the proposed method and its embodiments may be used for treatment of the human or animal body by surgery or therapy and may be a diagnostic method practiced on the human or animal body.

The making and usage of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference signs refer to the same technical features if not stated otherwise. As far as “may” is used in this application it means the possibility of doing so as well as the actual technical implementation. The present concepts of the present disclosure will be described with respect to preferred embodiments below in a more specific context namely heart and/or renal surgery and/or support (assist).

The disclosed concepts may also be applied, however, to other situations and/or arrangements in heart surgery/support and/or renal surgery/support as well, especially to surgery and/or support of other organs, e.g. brain, lung, abdomen, pancreas, digestive system, bladder, etc.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

For a more complete understanding of the presently disclosed concepts and the advantages thereof, reference is now made to the following description in conjunction with the accompanying drawings. The drawings are not drawn to scale. In the drawings the following is shown in:

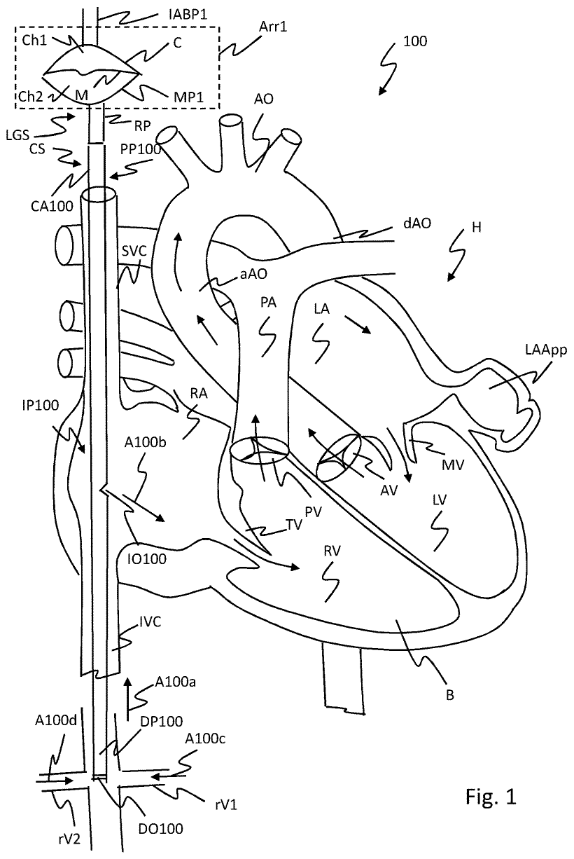

FIG. 1 a renal support (assist) system using a single membrane pump and a bidirectional cannula with blood delivery at an intermediate portion,

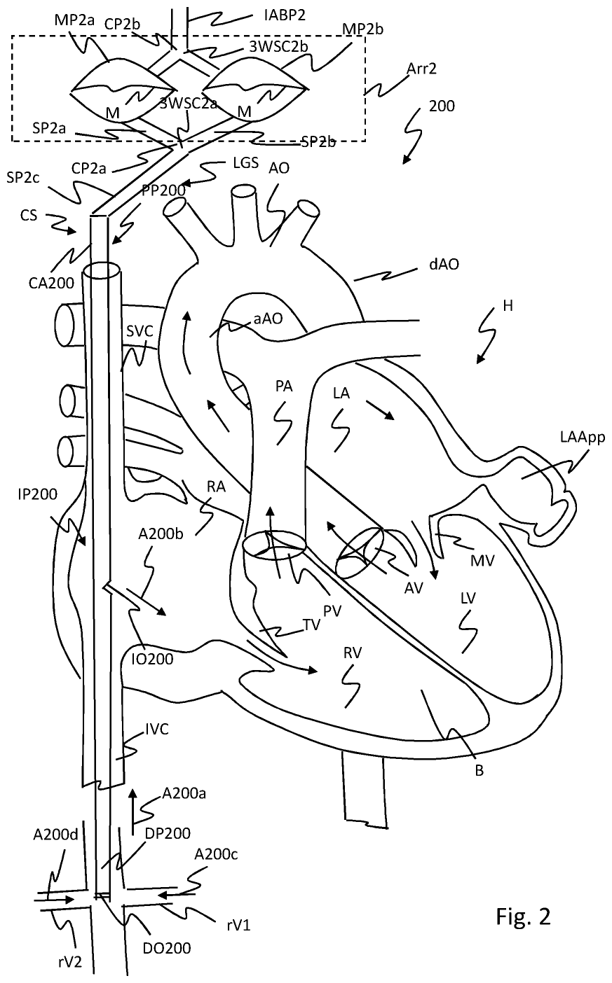

FIG. 2 a renal support (assist) system using two membrane pumps in parallel and a bidirectional cannula with blood delivery at an intermediate portion,

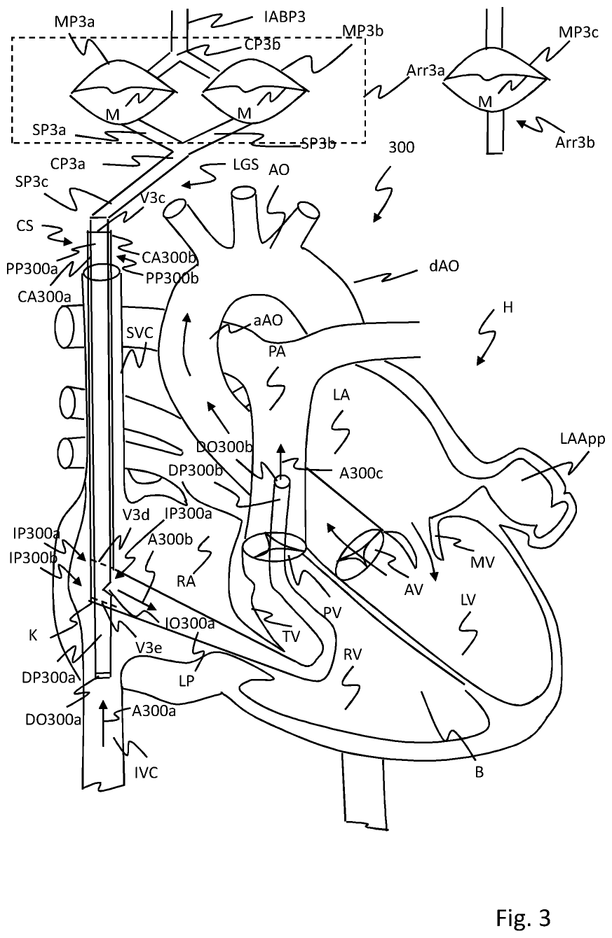

FIG. 3 a pRVAD® support (assist) system using two membrane pumps in parallel (dual membrane pump) or a single membrane pump,

FIG. 4 a pRVAD® support (assist) system using a pump arrangement comprising two membrane pumps in series with extracorporeal membrane oxygenation (ECMO) wherein a bidirectional cannula is used,

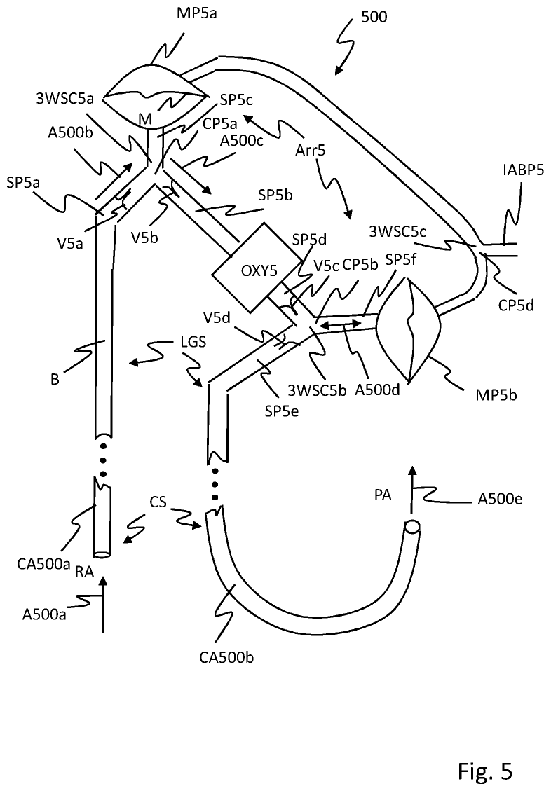

FIG. 5 a pRVAD® assist system using a pump arrangement comprising two membrane pumps in series with extracorporeal membrane oxygenation (ECMO) wherein two single lumen cannulas are used,

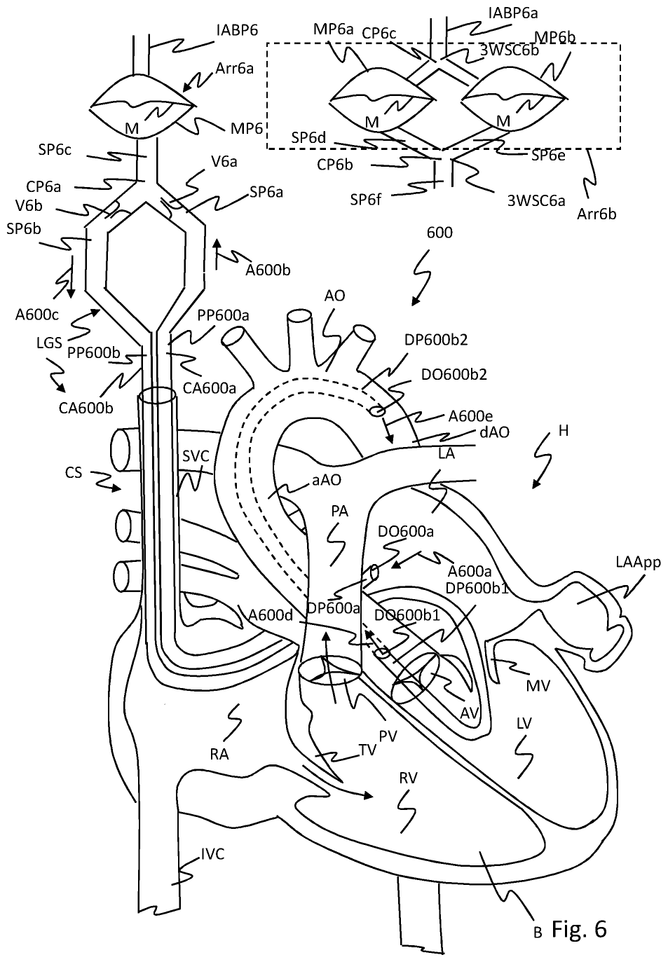

FIG. 6 a pLVAD® assist system with blood transport from left atrium to aorta, e.g. to ascending aorta or to descending aorta, with two cannulas,

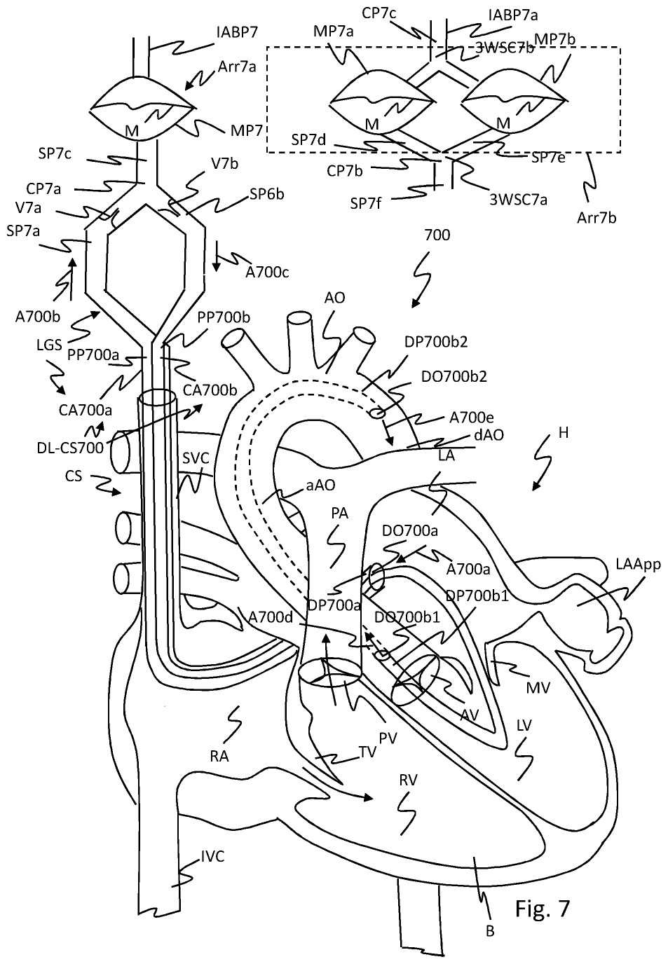

FIG. 7 a pLVAD® assist system with blood transport from left atrium to aorta, e.g. to ascending aorta or to descending aorta, with a dual lumen cannula system,

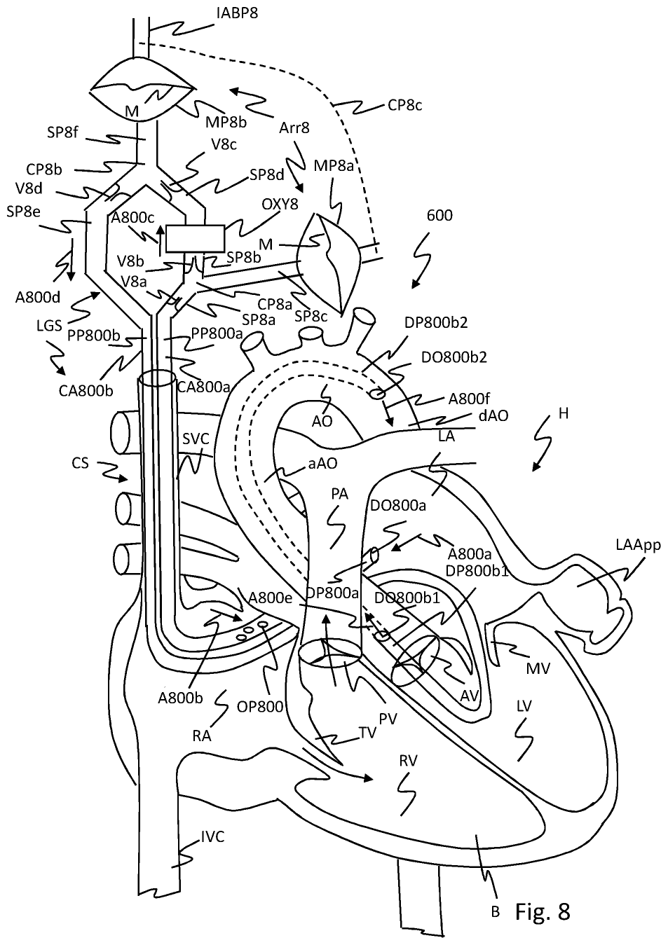

FIG. 8 a pBiVAD® assist system with blood transport from left atrium and right atrium to aorta, e.g. to ascending aorta or to descending aorta, with two cannulas,

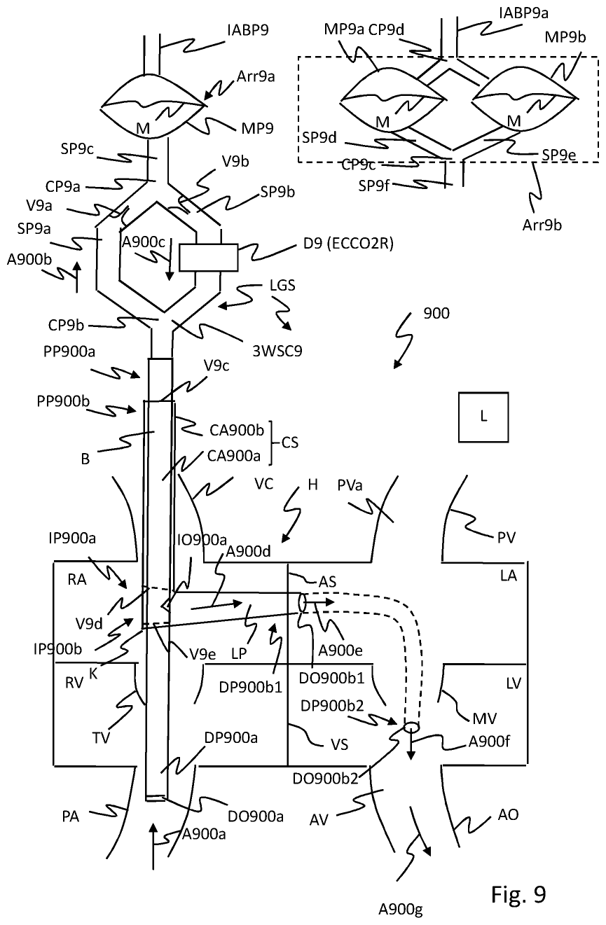

FIG. 9 an ECCO2R system with pulmonary drainage and delivery of blood into left atrium or into left ventricle using a bidirectional cannula and an outer cannula,

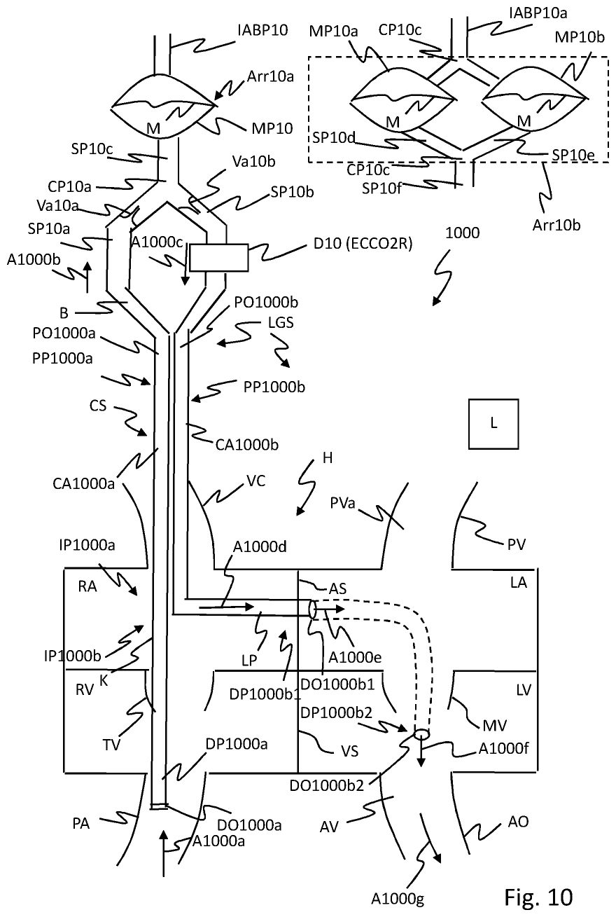

FIG. 10 an ECCO2R system with pulmonary drainage and delivery of blood into left atrium or into left ventricle using two separate or single lumen cannulas,

FIG. 11 a pBiVAD® assist system with blood transport from left atrium and right atrium to aorta, e.g. to ascending aorta or to descending aorta, using a dual lumen cannula system and one single lumen cannula (separate),

FIG. 11A a first embodiment of a pump arrangement for the pBiVAD® assist system of FIG. 11 including an oxygenator device,

FIG. 11B a second embodiment of a pump arrangement for the pBiVAD® assist system of FIG. 11 including an oxygenator device and allowing pulsatile outflow,

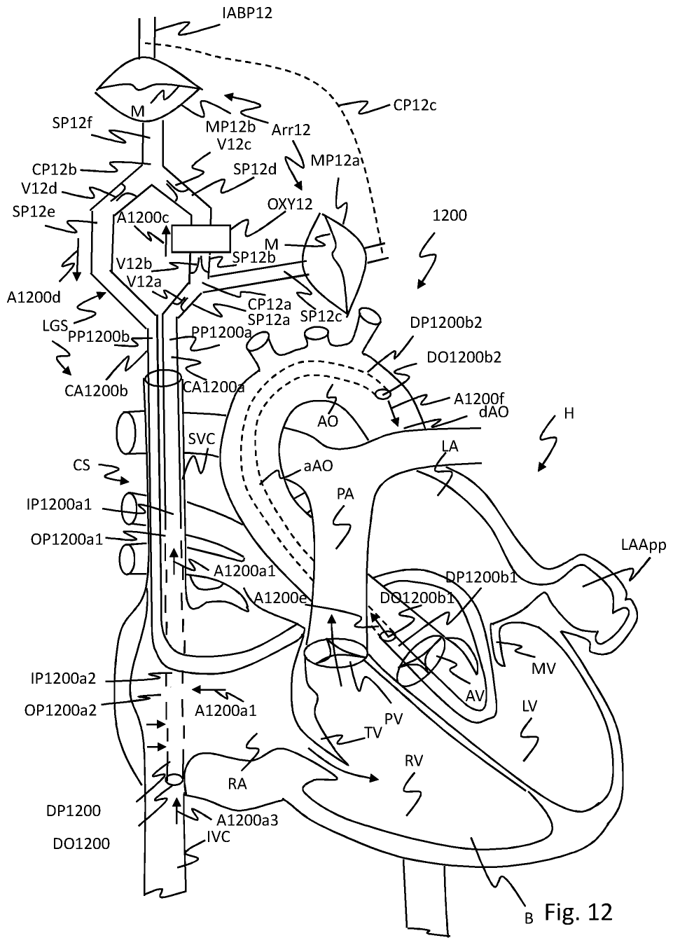

FIG. 12 a veno-arterial extracorporeal membrane oxygenation system with drain in right atrium and return cannula to aorta, e.g. to ascending aorta or to descending aorta, and

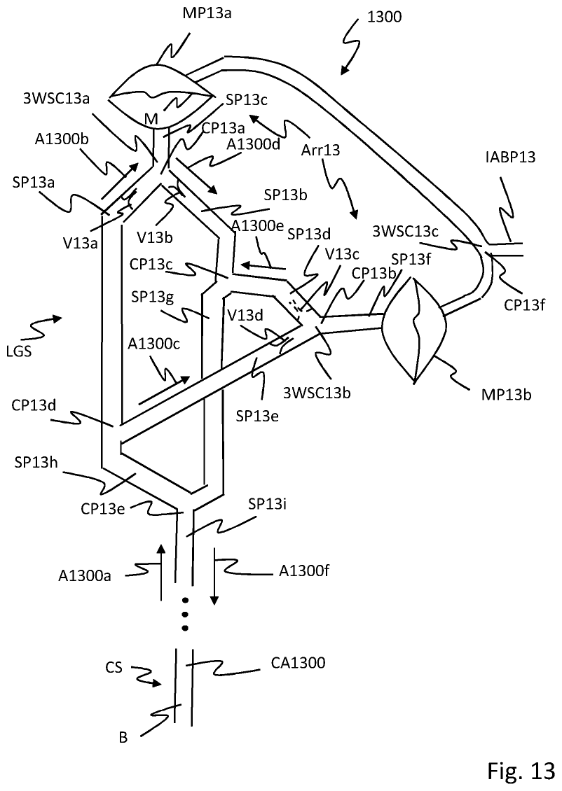

FIG. 13 a further arrangement comprising two membrane pumps operated in parallel.

FIG. 1 illustrates a renal support system 100 using a single membrane pump MP1 and a bidirectional cannula CA100 with blood delivery at an intermediate portion. Membrane pump MP1 as well as all other membrane pumps MP2 to MP12a, MP12b mentioned in the following may comprise:

a case C,

a membrane M within the case C, and

a reservoir port RP.

The membrane M may separate a reservoir chamber Ch1 within the case C from a compensation chamber Ch2 within the case C. Reservoir port RP may be connected to the reservoir chamber Ch1 and may be a bidirectional port, e.g. the only port of the reservoir chamber Ch1,

Furthermore a heart H is illustrated which comprises:

right atrium RA,

right ventricle RV,

left atrium LA,

left ventricle LV,

atrial septum AS between right atrium RA and left atrium LA, and

ventricle septum VS between right ventricle RV and left ventricle LV.

The following valves of heart H are shown in the following FIGS. 1 to 6:

tricuspid valve TV between right atrium RA and right ventricle RV,

mitral valve MV between left atrium LA and left ventricle LV,

aortic valve 155, AV is between aorta AO and left ventricle LV, and

pulmonary valve 150, PVa between right ventricle RV and pulmonary artery PA.

There are two left pulmonary veins 1PV and two right pulmonary veins rPV that extend into left atrium LA of heart H. Blood that is enriched with oxygen comes from lung L into left atrium LA through pulmonary veins PV. This is an exception in that a vein transports blood that comprises more oxygen than blood in a comparable artery. The description of heart H will not be repeated below. However, it is clear that this description is valid for all FIGS. 1 to 12 that show heart H.

FIG. 1 illustrates a renal support system 100 using a single membrane pump MP1 and a bidirectional cannula CA107 with blood delivery at an intermediate portion IP107.

Membrane pump MP1 comprises a case and a membrane M. The membrane M separates the case in two chambers, e.g. in a variable volume reservoir chamber Ch1 and in a compensations chamber Ch2 which may also be a driving chamber and which can compensate increased volume of the variable volume reservoir in chamber Ch1. An intra-aortic balloon pump console IABP1 may be used to drive membrane pump MP 1.

The distal portion of the bidirectional cannula CA100 may be inserted endovascularly, preferably jugularly, through superior vena cava SVC, right atrium RA and inferior vena cava IVC at least to a location which has to the junction of the renal veins rV1, rV2 into the inferior vena cava IVC a distance equal to 10 cm (centimeter) or less than 10 cm, equal to 5 cm or less than 5 cm or equal to 2.5 cm or less than 2.5 cm. Blood may be drained into the at least one distal opening DO100, see arrows A100a, A100c and A100d. Thereafter the blood is sucked to the membrane pump(s) MP1 in an aspiration phase. Blood is expulsed into cannula CA100 during an expulsion phase. The expulsed blood may be delivered out of the at least one intermediate opening IO100 of cannula CA100 into the right atrium RA, see arrow A100b.

Bidirectional cannula CA100 may be visible in an X-ray device or within another image generating device. Using the image generating device, the intermediated opening may be aligned such that it is directed to the center of the right atrium RA thus resulting in flow out of the intermediate opening I0100 which is directed directly to the tricuspid valve TV. A complete antegrad outflow is generated therewith, i.e. a blood flow which is directed in the natural flow directions of the blood. Opposite pulsation is avoided which results in less unnatural turbulences. The same may be valid for bidirectional cannula CA200 mentioned below.

Thus, blood may be pulled from the kidneys. Only one membrane pump MP1 may be used which may be coupled to an intra-aortic balloon pump console IABP1. IABP1 may be controlled by electric impulses of heart H.

FIG. 2 illustrates a renal support system 200 using two membrane pumps MP2a, MP2b in parallel and a bidirectional cannula CA200 with blood delivery at an intermediate portion IP200. All other parts correspond to parts mentioned in FIG. 1, e.g.:

cannula CA200 to cannula CA100,

proximal portion PP200 to PP100,

intermediate portion IP200 to IP100,

intermediate opening 10200 to I0100,

distal portion DP200 of cannula CA200 to distal portion DP100 of cannula CA100,

distal opening DO200 to DO100, and

arrows A200a to A200d to arrows A100a to A100d.

Thus, there is in principal the same function of renal support system 200 if compared with the function of renal support system 100 which is described above in detail. Differences in the function are described below.

Arrangement 200 as well as all other arrangements 300 to 1200 which are mentioned in the following may be used for transporting a liquid through a cannula system CS. Arrangement 200 to 1200 may comprise:

a liquid guiding system LGS which may be comprise:

at least three separated portions SP2a, SP2b, SP3c which define separate liquid guiding portions of the liquid guiding system LGS, and

a connecting portion CP2 which fluidically connects the at least three separated portions SP2a,

SP2b, SP3c and which comprises a lumen that branches out into at least two lumens.

The liquid guiding system LGS may be configured to be or may be connected to a pump arrangement Arr2 which drives a flow of the liquid. The liquid guiding system LGS may be configured to be or may be connected to a cannula system CS which is adapted to be inserted into a body of a human or of an animal and which comprises an inflow opening and an outflow opening of the liquid guiding system LGS.

In the Arrangement 200, 300, 600, 700, 900, 1000 there are two membrane pumps, for instance MP2a, MP2b which are operated in parallel and which are fluidically connected by a lower branch which forms an “Y”, e.g. which has a lower common portion. In this “Y” branch there is a first separated portion, for instance SP2a, SP3a, SP9d, of the at least three separated portions, for instance SP2a, SP2b, SP2c; SP3a, SP3b, SP3c; SP9d, SP9e, SP9f, and a second separated portion, for instance SP2b, SP3b, SP9e, of the at least three separated portions SP2a, SP2b, SP2c; SP3a, SP3b, SP3c; SP9d, SP9e, SP9f, etc. The first separated portion, for instance SP2a, SP3a, SP9d, and second separated portion, for instance SP2b, SP3b, SP9e, are configured to be or are connected to a pump arrangement Arr2, Arr3 which drives a flow of the liquid B, for instance blood, through the liquid guiding system LGS. The first separated portion, for instance, SP2a, SP3a, SP9d, is different from the second separated portion, for instance Sp2b, SP3b, SP9e. The arrangement, for instance 200, 300, 600, 700, 900, 1000, is configured such that a third separated portion, for instance SP2c, SP3c, SP9f, of the at least three separated portions, for instance SP2a, SP2b, SP2c; SP3a, SP3b, SP3c; SP9d, SP9e, SP9, is a common inlet and outlet portion through which flow flows coming from the first separated portion, for instance SP2a, SP3a, SP9d, and from the second separated portion, for instance SP2b, SP3b, SP9e, and/or through which flow flows which then flows to the first separated portion, for instance SP2a, SP3a, SP9d, and to the second separated portion, for instance SP2b, SP3b, SP9e.

Furthermore, there is an upper branch which connects two gas inlets of the at least two membrane pumps, for instance MP2a, MP2b. There is a connecting portion, for instance CP2b, which connects all separated portions of the branch. A common portion of the branch is connected to an intra-aortic balloon pump console IABP1, IABP2, etc. which is however not used to operate an intra-aortic balloon but to operate only one or at least two pumps, preferably membrane pumps. The two “lower” portions of the branch are connected to the connecting portion, for instance CP2b, and to each of the compensation chambers Ch2 of each membrane pump respectively, for instance to membrane pump MP2a and MP2b respectively. Air, helium or another gas is bidirectionally pushed into compensation chambers Ch2 (also drive chamber) and sucked out thereby pushing liquid/blood B out of the reservoir chamber Ch1 and sucking liquid/blood into reservoir chamber Ch1.

Three way stop cock members 3WSC2a, 3WSC2b, etc. may be used within connecting portions CP2a, CP2b. Alternatively, other valve members may be used instead of three way stop cock members 3WSC2a, 3WSC2b, etc. in order to fulfill the same or similar functions for allowing changing of the pump devices during operation.

FIG. 3 illustrates a pRVAD (percutaneous right ventricle assist device) support system 300 using two membrane pumps MP3a or MP3b in parallel (dual membrane pump) or a single membrane pump MP3c. Membrane pumps MP3a and MP3b may be coupled to the same port of an IABP console IABP3. IABP console IABP3 may be controlled by electrical signals of heart H. Both membrane pumps MP3a or MP3b may be connected to a proximal portion PP300a of a bidirectional cannula CA300a via a connecting portion CP3a and a separated portion SP3c.

Support system 300 may comprise a cannula system CS. The cannula system CS may comprise or may consist of the bidirectional cannula CA300a and an outer cannula CA300b. Bidirectional cannula CA300a may comprise:

a proximal portion PP300a,

an intermediate portion IP300a,

an intermediate opening IO300a,

a distal portion DP300a,

a distal opening DO300a, and

at least one internal valve or internal valve function.

Outer cannula CA300b may comprise:

a proximal portion PP300b,

a distal portion DP300b that comprises at least one distal opening DO300b,

a lumen portion LP that extends from the proximal portion PP300b to the at least one distal opening DO300b, and

at least one intermediate portion IP300b that is arranged between the proximal portion PP300b and the distal portion DP300b.

The intermediate portion IP300b of the outer cannula CA300b may comprise at least one intermediate opening IO300b which may be configured to allow passage of the distal portion DP300a of the bidirectional cannula CA300a.

Before inserting the bidirectional cannula CA300a into the body of the patient, the outer cannula CA300b is inserted through the vessels of the blood circuit. The distal portion DP300b of the outer cannula CA300b may be inserted endovascularly, preferably jugularly, through superior vena cava SVC, right atrium RA and right ventricle RV at least to pulmonary artery PA,

Thereafter, bidirectional cannula CA300a is inserted into outer cannula CA300b until the distal portion DP300a of bidirectional cannula CA300a extends through intermediate opening IO300b of outer cannula CA300b and intermediate opening IO300a of bidirectional cannula CA300a is arranged within intermediate portion IP300b of bidirectional cannula CA300a thereby being in fluidic connection with distal portion DP300b of the outer cannula CA300b. Distal portion DP300a of bidirectional cannula CA300a is inserted into right atrium RA optionally further into inferior vena cava IVC.

Blood B is drained into the at least one distal opening DO300a of bidirectional cannula CA300a, see arrow 300a. Blood B is transported in an aspiration phase into membrane pumps MP3a, MP3b; MP3c. In an expulsion phase blood B is transported in the opposite direction. Due to the valve(s) within bidirectional cannula CA300a or due to a specific fluidic design, blood is delivered out of the at least one intermediate opening I0300a of bidirectional cannula CA300a in the expulsion phase, see arrow A300b.

Blood is delivered further through the at least one distal opening DO300b of outer cannula CA300b, see arrow A300c. Usage of an oxygenator is optional in pVRAD system 300.

Although outer cannula CA300b is illustrated with different diameters, especially in intermediate portion IP300b it is of course also possible to have a constant diameter along the longitudinal axis of outer cannula CA300b.

Furthermore, outer cannula CA300b may have a kink K in intermediate portion IP300b. In a state without outer forces (base state) kink K may include an angle in the range of 80 degrees to 130 degrees, preferably 110 degrees. Kink K may facilitate the insertion of distal portion DP300a of bidirectional cannula CA300a through intermediate opening I0300b of outer cannula CA300b. However, it is also possible to use an outer cannula without a kink K.

Bidirectional cannula CA300a may be essentially straight or straight if no external forces are applied. This may be true for all other embodiments of bidirectional cannulas mentioned in this description, for instance bidirectional cannula CA900a which is mentioned below.

With regard to valves V3c, V3d and V3e see description at the end of the description of FIG. 9.

Arrangement 300 may comprise a pump arrangement Arr3 which is similar to pump arrangement Arr2 described above in detail. Thus, it is again possible to change one of the membrane pumps MP3a or MP3b during operation of arrangement 300. Alternatively, only one membrane pump MP3c may be used, for instance if less pumping volume is necessary and/or if changing of a pump device may not be necessary.

FIG. 4 illustrates a pRVAD R support (assist) system using a pump arrangement Arr4 comprising two membrane pumps MP4a, MP4b in series with extracorporeal membrane oxygenation (ECMO) wherein a bidirectional cannula CA300, see FIG. 3, or a bidirectional cannula CA 400 for another medical application is used.

In arrangement 400 as well as in arrangements 500 to 1200 there is at least one membrane pump, for instance MP4a but also MP5a, which is fluidically connected to a common portion, for instance SP4c, SP4f, which branches into two separated portions, for instance separated portion SP4a and separated portion SP4b for separated portion SP4c. See also separated portion SP4d and separated portion SP4e for separated portion SP4f. The branch may have the form of a “Y” rotated by 180 degrees, see for instance separated portions SP4a, SP4b and SP4c.

Within these rotated “Y” branches there is a first separated portion, for instance SP4a, of the at least three separated portions, for instance SP4a, SP4b, SP4c, which is an inflow portion of the liquid guiding system LGS. A second separated portion, for instance SP4b, of the at least three separated portions, for instance SP4a, SP4b, SP4c, is an outflow portion of the liquid guiding system LGS. The first separated portion, for instance SP4a, is different from the second separated portion, for instance SP4b.

A third separated portion, for instance SP4c, of the at least three separated portions SP4a, SP4b, SP4c, is configured to be connected to a pump arrangement, for instance Arr4 which drives a flow of the liquid/blood B. Arrangement 400 but also arrangements 500 to 1200 are configured such that the third separated portion, for instance SP4c, is a portion through which flow coming from the first separated portion, for instance SP4a, flows and/or through which third separated portion, for instance SP4c, flow flows which flows then to the second separated portion, for instance SP4b. However, no fluid flow may flow directly from first separated portion, for instance SP4a, to second separated portion, for instance SP4b.

There may be the following one-way valves in arrangement 400:

a one-way valve V4a within first separated portion SP4a,

a one-way valve V4b within second separated portion SP4b,

a one-way valve V4c within separated portion SP4d, and

a one-way valve V4d within separated portion SP4e.

One-way valves V4a to V4d are symbolized by “arrows” which to not necessarily correspond to the internal structure of these valves. However, the direction of the “arrow” corresponds to the flow direction which is possible through the respective valve V4a to V4d.

Arrangement 400 may further comprise an oxygenator device OXY4 which may enhance blood B with oxygen. Alternatively or additionally, other blood treatment devices may be used, for instance blood B filter, e.g. dialysis devices, heating or cooling devices, medicament delivery devices etc.

Thus, arrangement 400, as well as other arrangements e.g. 500, 800, 1200, is configured such that the outflow of only one pump device, for instance of pump MP4a, MP5a, MP8a, MP12a, of the at least two pump devices, for instance MP4a, MP4b; MP5a, MP5b; MP8a, MP8b; MP12a, MP12b, flows through the oxygenator device, for instance OXY4, OXYS, OXY8, OXY12. The output of the other pump device MP4b, MP5b, MP8b, MP12b does not flow through the oxygenator device, for instance OXY4, OXYS, OXY8, OXY12. The outflow of the oxygenator device, for instance OXY4, OXYS, OXY8, OX12, flows through the other pump device MP4b, MP5b, MP8b, MP12b of the at least two pump devices MP4a, MP4b; MP5a, MP5b; MP8a, MP8b; MP12a, MP12b resulting in a pulsatile output flow.

In more detail, blood may be sucked into arrangement 400, see arrow A400a. Thereafter, the blood B flows through separated portion SP4a and one-way valve V4a and further through separated portion SP4c into membrane pump MP4a, see arrow A400b. Membrane pump MP4a expulses the blood B in an expulsion phase. The expulsed blood is blocked by one-way valve V4a but may flow through portion SP4c and portion SP4b (through one-way valve V4b), see arrow A400c. Blood B flows then through the oxygenator device OXY4 where it is enriched with oxygen. After oxygenation blood B flows through separated portion SP4d (including valve V4c) into membrane pump MP4b in an aspiration phase, e.g. there is a pressing of flow into the oxygenator device at the inflow port of the oxygenator device and a sucking of flow at the outflow port of the oxygenator device which may enhance the overall throughput through the oxygenator device, for instance oxygenator device OXY4. Membrane pump MP4b expulses blood B which is pressed though separated portion SP4f into separated portion SP4e, see arrow A400d. Expulsed blood is blocked by one-way valve V4c. Finally the blood flows out of separated portion SP4e via a connecting portion CP4c into cannula CA300a or into another cannula CA400, see arrow A400e. Connecting portion CP4c connects separated portion SP4a and SP4e forming a closed fluid guide loop. However, within this loop fluid may flow only in one direction because of the one-way valves V4a to V4d.

Membrane pumps MP4a and MP4b are connected to an IABP console IABP4 at their gas inlets. A further connecting portion CP4d connects the air ports of membrane pump MP4a and MP4b to IABP console IABP4.

A connecting portion CP4a is between separated portions SP4a, SP4b and SP4c. A connecting portion CP4b is between separated portions SP4d, SP4e and SP4f.

Tree way stop cocks 3WSC4a to 3WSC4c may be used at connecting portions CP4a, CP4b and CP4c in order to allow easy change of membrane pump MP4a and/or MP4b during operation of arrangement 400.

It is for instance possible to omit some of the one-way valves, for instance valve V4b. It may be possible to omit both one-way valves V4b and V4c if oxygenator device OXY4 fulfills a valve function. The same may be true for the valves illustrated in FIGS. 5, 8 and 12.

FIG. 5 illustrates a pRVAD R assist system using a pump arrangement Arr5 comprising two membrane pumps MP5a, MP5b in series with extracorporeal membrane oxygenation (ECMO) wherein two single lumen cannulas CA500a and CA 500b are used. A distal portion of cannula CA500a may be arranged in right atrium RA and/or in superior vena cava SVC and/or inferior vena cava IVC. A distal portion of cannula CA500b may be arranged in pulmonary artery PA.

Other parts of the arrangement 500 which is illustrated in FIG. 5 correspond to parts mentioned in FIG. 4, e.g.:

separated portions SP5a to SP5f to separated portions SP4a to SP4f respectively,

connecting portions CP5a, CP5b and CP5d to connecting portions CP4a, CP4a and CP4d,

oxygenator device OXY5 to oxygenator device OXY4,

one-way valves V5a to V5d to one-way valves V4a to V4d respectively, and

IABP console IAPB5 to IABP4.

The main difference between arrangement 500 and arrangement 400 is that connecting portion CP4c is not realized in arrangement 500, e.g. cannula CA500a is coupled or connected only to separated portion SP5a but not to separated portion SP5e. Cannula CA500b is coupled or connected only to separated portion SP5e but not to separated portion SP5a.

The function of both arrangements 400, 500 is however, similar, e.g.:

arrow A500a illustrates an inflow in cannula CA500a,

arrow A500b illustrates directed flow through separated portion SP5a into membrane pump MP5a,

arrow A500c illustrates expulsion flow from membrane pump MP5a to oxygenator device OXAS through separated portion SP5b,

arrow A500d illustrates flow into membrane pump MP5b from separated portion SP5d and out of membrane pump MP5a through separated portion SP5e, and

arrow A500e illustrates an outflow at distal portion of single lumen cannula CA500b.

Again three way stop cocks 3WSC5a to 3WSC5c or other elements may be used in order to enable change of membrane pumps MP5a, MP5b during operation.

FIG. 6 illustrates a pLVADR assist system or arrangement 600 with blood B transport from left atrium LA to aorta AO, e.g. to ascending aorta aAO or to descending aorta dAO, with two single lumen cannulas CA600a and CA600b. Cannula CA600a comprises:

a proximal portion PP600a,

a distal portion DP600a, and

a distal opening DO600a within distal portion DP600a.

Distal opening DO600a is arranged within left atrium LA.

Cannula CA600b comprises in a first variant:

a proximal portion PP600b,

a distal portion DP600b1, and

a distal opening DO600b1 within distal portion DP600b1.

Distal opening DO600b1 is arranged within ascending aorta aAO.

Cannula CA600b comprises in a second variant:

a proximal portion PP600b,

a distal portion DP600b2, and

a distal opening DO600b2 within distal portion DP600b2.

Distal opening DO600b2 is arranged within descending aorta dAO.

Cannula CA600a may be inserted through one of the jugular veins, through superior vena cava SVC, through right atrium RA, atrial septum AS into left atrium LA. Cannula CA600b may be inserted through one of the jugular veins, through superior vena cava SVC, through right atrium RA, through atrial septum AS, through left atrium LA, through left ventricle LV and further into aorta AO.

Arrangement 600 comprises a pump arrangement Arr6a comprising only one membrane pump MP6a which is driven by an IABP console IABP6.

Arrangement 600 comprises further:

a separated portion SP6a which is connected with proximal portion PP600a of cannula CA600a and with a connecting portion CP6a,

a separated portion SP6b which is connected with proximal portion PP600b of cannula CA600b and with connecting portion CP6a,

a separated portion SP6c which is connected with liquid flow port (reservoir port RP) of membrane pump MP6 and with connecting portion CP6a.

There may be the following one-way valves in arrangement 600:

a one-way valve V6a within separated portion SP6a, and

a one-way valve V6b within separated portion SP6b.

The function of arrangement 600 is as follows:

an arrow A600a illustrates inflow in cannula CA600a from left atrium LA,

an arrow A600b illustrates inflow through separated portion SP6a and through one-way valve V6a,

an arrow A600c illustrates outflow through separated portion SP6b and through one-way valve V6b into cannula CA600b,

an arrow A600d illustrates outflow through distal opening DO600b1 (variant 1), and

an arrow A600e illustrates outflow through distal opening DO600b2 (variant 2).

Alternatively, a pump arrangement Arr6b may be used instead of pump arrangement Arr6a which comprises only one membrane pump MP6. Pump arrangement Arr6b comprises:

membrane pumps MP6a and MP6b,

a connecting portion CP6b,

a (first in original claim 2) separated portion SP6d which is connected to the liquid flow port of membrane pump MP6a,

a (second in original claim 2) separated portion SP6e which is connected to the liquid flow port of membrane pump MP6b,

a (third in original claim 2) separated portion SP6f which is connected to connecting portion SP6c, and

a connecting portion CP6c, which corresponds to connecting portion CP2b mentioned above, e.g. it is between gas ports of membrane pumps MP6a and MP6b and an IABP console IABP6a.

Connecting portion CP6b is arranged between separated portions SP6d to SP6f. Connecting portion CP6b may be a Y-connector or a T-connector or another 3 port element. Alternatively an X-connector may be used which comprises both connecting portions CP6a and CP6b.

Three way stop cocks 3WSC6a, 3WSC6b may be used in pump arrangement Arr6b in order to ease changing of at least one of the membrane pump MP6a, MP6b during operation of arrangement 600 and preferably also of pump arrangement Arr6b.

FIG. 7 illustrates apLVADR assist system (arrangement) 700 with blood B transport from left atrium LA to aorta AO, e.g. to ascending aorta aAO or to descending aorta dAO, with a dual lumen cannula system DL-CS700.

Other parts of the arrangement 700 which is illustrated in FIG. 7 correspond to parts mentioned in FIG. 6, e.g.:

separated portions SP7a to SP7f to separated portions SP6a to SP6f respectively,

connecting portions CP7a to CP7c to connecting portions CP6a to CP6c,

one-way valves V7a and V7b to one-way valves V6a and V6b respectively, and

IABP console IAPB7, IABP7a to IABP6, IABP6a.

There are the following differences:

an outer cannula CA700a of dual lumen cannula system DL-CS700 replaces cannula CA600a,

an inner cannula CA700b of dual lumen cannula system DL-CS700 replaces cannula CA600b,

proximal portion PP700a of outer cannula CA700a is connected with separated portion SP7a, and

proximal portion PP700b of inner cannula CA700b is connected with separated portion SP7b.

Both cannulas CA700a, CA700b of dual lumen cannula system DL-CS700 are inserted through the atrial septum. Outer cannula CA700a may be inserted first the same way as cannula CA600a. Thereafter, inner cannula CA700b may be inserted into outer cannula CA700a to left atrium LA and then further as described above for cannula CA600b, e.g. there may be again two variants (variant 1 and variant 2).

The function of arrangement 700 is as follows:

an arrow A700a illustrates inflow in outer cannula CA700a from left atrium LA,

an arrow A700b illustrates inflow through separated portion SP7a and through one-way valve V7a,

an arrow A700c illustrates outflow through separated portion SP7b and through one-way valve V7b into cannula CA700b,

an arrow A700d illustrates outflow through distal opening DO700b1 (variant 1), and

an arrow A00e illustrates outflow through distal opening DO700b2 (variant 2).

Alternatively, a pump arrangement Arr7b may be used instead of pump arrangement Arr7a. Pump arrangement Arr7b corresponds to pump arrangement Arr6b, see detailed description above.

FIG. 8 illustrates a pBiVAD assist system with blood transport from left atrium LA and right atrium

RA to aorta AO, e.g. to ascending aorta aAO or to descending aorta dAO, with two cannulas CA800a and CA800b.

Other parts of the arrangement 800 which is illustrated in FIG. 8 correspond to parts mentioned in FIG. 5, e.g.:

separated portions SP8a to SP8e to separated portions SP5a to SP5e respectively,

connecting portions CP8a to CP8c to connecting portions CP5a to CP5c,

oxygenator device OXY8 to oxygenator device OXY5,

one-way valves V8a to V8d to one-way valves V5a to V5d respectively, and

IABP console IAPB8 to IABP5.

Cannulas CA800a comprises:

a proximal portion PP800a,

a distal portion DP800a, and

a distal opening DO800a within distal portion DP800a.

Distal opening DO800a is arranged within left atrium LA.

Cannula CA800b comprises in a first variant (variant 1):

a proximal portion PP800b,

a distal portion DP800b1, and

a distal opening DO800b1 within distal portion DP800b1.

Distal opening DO800b1 is arranged within ascending aorta aAO.

Cannula CA800b comprises in a second variant (variant 2):

a proximal portion PP800b,

a distal portion DP800b2, and

a distal opening DO800b2 within distal portion DP800b2. Distal opening DO800b2 is arranged within descending aorta dAO.

Cannula CA800a may be inserted through one of the jugular veins, through superior vena cava SVC, through right atrium RA, atrial septum AS into left atrium LA. Cannula CA800a comprises openings

OP800 which are located in right atrium RA in the inserted state of cannula CA800a.

Cannula CA800b may be inserted through one of the jugular veins, through superior vena cava SVC, through right atrium RA, through atrial septum AS, through left atrium LA, through left ventricle LV and further into aorta AO.

The function of arrangement 800 is as follows:

an arrow A800a illustrates an inflow into distal opening DO800a of cannula CA800a,

an arrow A800b illustrates a further inflow into openings OP800 of cannula CA800a,

an arrow A800c illustrates blood flow through oxygenator device OXY8 after blood has been pumped by membrane pump MP8a,

an arrow A800d illustrates blood flow within separated portion SP8e after it has been pumped by membrane pump MP8b, and

an arrow A800e illustrates an outflow at distal portion DP800b1 of cannula CA800b (variant 1), and

an arrow A800f illustrates an alternative outflow at distal portion DP800b2 of cannula CA800b (variant 2).

FIG. 9 illustrates an ECCO2R system 900 with pulmonary artery PA drainage and delivery of blood B into left atrium LA or into left ventricle LV. A cannula system CS may be used that comprises two cannulas CA900a and CA900b. A bidirectional cannula CA900a may comprise:

a proximal portion PP900a,

an intermediate portion IP900a,

an intermediate opening IO900a,

a distal portion DP900a,

a distal opening DO900a, and

at least one internal valve or internal valve function.

An outer cannula CA900b may comprise:

a proximal portion PP900b,

a distal portion DP900b1 (variant 1), DP900b2 (variant 2) that comprises at least one distal opening DO900b1 or DP900b2,

a lumen portion LP that extends from the proximal portion PP900b to the at least one distal opening DO900b1 or DP900b2, and

at least one intermediate portion IP900b that is arranged between the proximal portion PP900b and the distal portion DP900b1 or DP900b2.

The intermediate portion IP900b of the outer cannula CA900b comprises at least one intermediate opening IO900b which is configured to allow passage of the distal portion DP900a of the bidirectional cannula CA900a.

Again, the outer cannula CA900b may be inserted before bidirectional cannula CA900a is inserted into the body of the patient. In a first alternative, the distal portion DP900b1 of the outer cannula CA900b is inserted endovascularly, preferably jugularly, through superior vena cava SVC, right atrium RA and atrial septum AS up to the left atrium LA of the heart H.

In a second alternative, the distal portion DP900b2 of the outer cannula is CA900b is inserted endovascularly, preferably jugularly, through superior vena cava SVC, right atrium RA and atrial septum AS up to the left atrium LA of the heart H and then further through mitral valve MV into left ventricle LV. In a further alternative which is not illustrated the distal portion DP900b2 of the outer cannula CA900b is inserted further, for instance up to the ascending aorta AO.

After insertion of the outer cannula CA900b, e.g. after the outer cannula CA900b is in place, the distal portion DP900a of the bidirectional cannula CA900a is inserted through the proximal portion PP900b of the outer cannula CA900b, through the intermediate portion IP900b of the outer cannula CA900b, through the intermediate opening IO900b of the outer cannula CA900b, into the right atrium RA, through the right ventricle RV and at least to or up to the pulmonary artery PA.

At least one membrane pump MP9 may be connected with the proximal end of the bidirectional cannula CA900a and blood B may be drained into the at least one distal opening D0900 of the bidirectional cannula CA900a in an aspiration phase. In an expulsion phase of the membrane pump MP9 operation, blood B is delivered out of the at least one intermediate opening 10900a of the bidirectional cannula CA900a and further through the at least one distal opening DO900b1 of the outer cannula CA900b into left atrium LA in the first alternative. In the second alternative, blood is delivered out of the at least one intermediate opening IO900a of the bidirectional cannula CA900a and further through the at least one distal opening DO900b2 of the outer cannula CA900b into left ventricle LV.

The drained blood B may be enriched in all alternatives with oxygen and/or it may be depleted from carbon dioxide outside of the body of a patient before it is delivered out of the intermediate opening IO900a of the bidirectional cannula CA900a. An ECCO2R (extracorporeal carbon dioxide removal) system may be used which may have lower pressures and/or throughput rates (volume per minute) compared to the usage of an oxygenator, especially within an ECMO (extracorporeal membrane oxygenation). However, both blood treatment methods are optionally.

Arrangement 900 comprises a pump arrangement Arr9a comprising only one membrane pump MP9a which is driven by an IABP console IABP9.

Arrangement 900 comprises further:

a separated portion SP9a which is connected with proximal portion PP900a of cannula CA900a via a further connecting portion CP9b and with a connecting portion CP9a,

a separated portion SP6b which is connected with an input port of a device D9 for carbon dioxide removal (ECCO2R) and with connecting portion CP9a,

a separated portion SP9c which is connected with liquid flow port (reservoir port RP) of membrane pump MP9 and with connecting portion CP9a,

a further portion which is connected to an output (outflow) port of device D9 and to connecting portion CP9b.

Connecting portion CP9b is also connected to proximal portion PP900a of bidirectional cannula CA900a.

Thus, there is a closed loop fluid guide within arrangement 900.

There may be the following one-way valves in arrangement 900:

a one-way valve V9a within first separated portion SP9a, and

a one-way valve V9b within separated portion SP9b.

The one-way valves V9a, V9b may provide a directed flow in only one direction within the closed fluid guide loop in arrangement 900.

The function of arrangement 900 is as follows:

an arrow A900a illustrates inflow of blood B into cannula CA900a from pulmonary artery PA,

an arrow A900b illustrates flow through separated portion SP9a and through one-way valve V9a,

an arrow A900c illustrates outflow through separated portion SP9b and through one-way valve V9b into device D9 (ECCO₂R),

an arrow A900d illustrates outflow through intermediate opening 10900a of bidirectional cannula CA900a into a lumen of outer cannula CA900b,

an arrow A900e illustrates outflow through distal opening DO900b1 (variant 1) into left atrium LA, and

an arrow A900f illustrates outflow through distal opening DO900b2 (variant 2) into left ventricle LV.

Alternatively, a pump arrangement Arr9b may be used instead of pump arrangement Arr9a which comprises only one membrane pump MP9. Pump arrangement Arr9b comprises:

membrane pumps MP9a and MP9b,

a connecting portion CP9c,

a (first in original claim 2) separated portion SP9d which is connected to the liquid flow port of membrane pump MP9a,

a (second in original claim 2) separated portion SP9e which is connected to the liquid flow port of membrane pump MP9b,

a (third in original claim 2) separated portion SP9f which is connected to connecting portion SP9c, and

a connecting portion CP9d, which corresponds to connecting portion CP2b mentioned above, e.g. it is between gas ports of membrane pumps MP9a and MP9b and an IABP console IABP9a.

Connecting portion CP9c is arranged between separated portions SP9d to SP9f. Connecting portion CP9c may be a Y-connector or a T-connector or another 3 port element. Alternatively an X-connector may be used which comprises both connecting portions CP9a and CP9c.

Three way stop cocks may be used in pump arrangement Arr9b in order to ease changing of at least one of the membrane pump MP9a, MP9b during operation of arrangement 900 and preferably also of pump arrangement Arr9b.

Furthermore, valves V3c to V3e, V9c to V9e or other sealing elements may be used, for instance multi-flap valves or another self-sealing member (for instance a simple sealing ring), i.e. for instance two flexible membranes. Other types of hemostasis valves may also be used.

Valve V3c, V9c may prevent that blood flows out of the proximal portion PP300b, PP900b of the outer cannula CA300b, CA900b, especially if the bidirectional cannula CA300a, Ca900a is not yet in the inserted state within outer cannula CA300b, CA900b. A multi-flap valve may be used for valve V3c, V9c.

Valve V3d, V9d may prevent that blood flows into the space or “dead” lumen between intermediate portion IP300b, IP900b and thus into a possible space between both cannulas CA300a, CA300b or CA900a, CA900b. This may result in preventing clotting of the blood in regions of the cannula system CS where the blood flow may be not high enough. A multi-flap valve may be used for valve V3d, V9d. Alternatively, a sealing ring or other sealing member may be used for valve V3d, V9d.

Valve V3e, V9e may be used to prevent that blood which is delivered out of intermediate opening I0300a, IO900a of bidirectional cannula CA300a, CA900a flows out of intermediate opening IO300b, IO900b of outer cannula CA300a, CA900b and thus in regions in which it should not be flow, i.e. the complete delivery flow may reach the distal opening DO300b, DO900b1 or DO900b2. If valve V3d, V9d is used valve V3e, V9e may be a simple sealing ring. However, other types of valves may also be used for valves V3e, V9e. A multi-flap valve may be used for valve V3e, V9e.

Valves V3d, V3e, V9d and V9e make sure that blood that flows out of intermediate opening IO300a, IO900a flows within outer cannula CA300b, CA900b to the distal opening DO300b, DO900b.

Although outer cannula CA900b is illustrated with different diameters, especially in the intermediate portion IP900b it is of course also possible to have a constant diameter along the longitudinal axis of outer cannula CA900b.

Furthermore, outer cannula CA900b may have a kink K in the intermediate portion. In a state without outer forces (base state) the kink K may include an angle in the range of 80 degrees to 130 degrees, preferably 110 degrees. Kink K may facilitate the insertion of the distal portion DP900a of bidirectional cannula CA900a through intermediate opening IO900b of outer cannula CA900b. However, it is also possible to use an outer cannula without a kink K.

FIG. 10 illustrates an ECCO2R system 1000 with pulmonary artery PA drainage and delivery of blood into left atrium LA or into left ventricle LV using two separate or single lumen cannulas CA1000a and CA1000b each comprising a proximal portion PP1000a, PP1000b, a distal portion DP1000a, DP1000b and a distal opening DO1000a, DO1000b in distal portion DP1000a, DP1000b. Proximal portion

PP1000a, PP1100b comprises a proximal opening PO1000a, PO1000b respectively.

Other parts of the arrangement 1000 which is illustrated in FIG. 10 correspond to parts mentioned in FIG. 9, e.g.:

separated portions SP10a to SP1Of to separated portions SP9a to SP9f respectively,

connecting portions CP10a to CP10c to connecting portions CP9a to CP9c,

one-way valves V10a and V10b to one-way valves V9a and V9b respectively,

device D10 to device D9, e.g. ECCO₂R, and

IABP console IAPB10, IABP10a to IABP9, IABP9a.

There are the following differences:

there is no closed loop because separated portion SP10a is connected only to proximal portion PP1000a of cannula CA1000a,

a portion at the output of device D10 is connected only to proximal portion PP1000b of cannula CA1000b, and

there is no connecting portion which corresponds to connecting portion CP9b.

The function of arrangement 1000 is as follows:

an arrow A1000a illustrates inflow in cannula CA1000a from pulmonary artery PA,

an arrow A1000b illustrates flow through separated portion SP10a and through one-way valve V10a,

an arrow A1000c illustrates outflow through separated portion SP10b and through one-way valve V10b into device D10 (ECCO2R),

an arrow A1000d illustrates flow within cannula CA1000b,

an arrow A900e illustrates outflow through distal opening DO1000b1 (variant 1) into left atrium LA, and

an arrow A900f illustrates outflow through distal opening DO1000b2 (variant 2) into left ventricle LV.

Alternatively, a pump arrangement Arr10b may be used instead of pump arrangement Arrl0a which comprises only one membrane pump MP9. Pump arrangement Arr10b corresponds to pump arrangement Arr9b, see description above.

Optionally, three way stop cocks may be used in arrangement 1000 as well.

FIG. 11 illustrates a pBiVAD R assist system or arrangement 1100 with blood B transport from left atrium LA and right atrium RA to aorta AO, e.g. to ascending aorta aAO or to descending aorta dAO, using a dual lumen cannula system DL-CS1000 and one single lumen cannula CA1100c which is a separate cannula.

Dual lumen cannula system DL-CS1000 comprises:

an inner cannula CA1100a, and

an outer cannula CA1100b.

Inner cannula CA1100a comprises:

a proximal portion PP1100a,

a distal portion DP1100a, and

a distal opening D01100a within distal portion DP1100a.

Distal opening D01100a is arranged within left atrium LA.

Outer cannula CA1100b comprises:

a proximal portion PP1100b,

a distal portion DP1100b, and

a distal opening D01100b or several distal openings D01100b within distal portion DP1100b.

Distal opening(s) D01100b is (are) arranged within right atrium RA.

Outer cannula CA1100b may be inserted through superior vena cava SVC into right atrium RA. Thereafter, inner cannula CA1100a may be inserted into outer cannula CA1100b and further through right atrium RA, through atrial septum AS into left atrium LA.

Cannula CA1100c may be inserted through superior vena cava SVC into right atrium RA and then transseptal through atrial septum AS, through left atrium LA, through left ventricle LV into ascending aorta aAO (variant 1) or up to descending aorta dAO (variant 2).

The function of arrangement 1100 is as follows:

an arrow A1100a illustrates inflow into inner cannula CA1100a from left atrium LA,

an arrow Al 100b illustrates outflow from proximal portion PP1100a of inner cannula CA1100a, see FIGS. 11A and 11B,

an arrow A1100c illustrates inflow into distal opening(s) of outer cannula CA1100b from right atrium RA,

an arrow A1100d illustrates outflow from proximal portion PP1100b of outer cannula CA1100b, see FIGS. 11A and 11B,

an arrow A1100d illustrates inflow into proximal portion PP1100c of single lumen cannula CA1100c, see FIGS. 11A and 11B,

an arrow A1100f illustrates outflow through distal opening D01100b1 (variant 1) of single lumen cannula CA1100c into ascending aorta aAO, and

an arrow A1100g illustrates outflow through distal opening D01100b2 (variant 2) of single lumen cannula CA1100c into descending aorta dAO.

Two variants of pump arrangements for arrangement 1000 are illustrated in FIG. 11A and in FIG. 11B respectively.

FIG. 11A illustrates a first embodiment of a pump arrangement ArrllA for the pBiVADR assist system or arrangement 1100, 1100A of FIG. 11 including an oxygenator device OXY11A.

Arrangement 1100A comprises:

a pump arrangement Arr11A, and

the oxygenator device OXY11A.

Pump arrangement ArrllA may comprise:

a membrane pump MP11Aa and a membrane pump MP1 lAb,

a connecting portion CP1 lAc which connects gas ports of membrane pump MP11Aa and a membrane pump MP11Ab with an IABP console IAPB11A.

A single liquid flow port of membrane pump MP11Aa is connected to a separate portion SP11Ac. A connecting portion CP1 lAa is between separated portion SP11Ac and two further separated portions

SP11Aa, SP11Ab. Separated portion SP11Aa is connected with the proximal portion PP1100b of cannula CA1100b. Separated portion SP11Ab is connected with a connecting portion CP11Ad.

Connecting portion CP1 lAd is furthermore fluidically connected to an input port of oxygenator device OXY11A via a portion P11A1 as well as to a separate portion SP11Ad.

A single liquid flow port (RP) of membrane pump MP1 lAb is connected to a separate portion SP11Af. A connecting portion CP1 lAb is between separated portion SP11Af and two further separated portions SP11Ad, SP11Ae. Separated portion SP11Ad is connected with connecting portion CP1 lAd as already mentioned. Separated portion SP11Ae is connected with proximal portion PP1100a of cannula CA1100a..

The output port of oxygenator device OXY11A is connected to a portion P11A2. Portion P11A2 is fluidically connected to proximal portion PP1100c of cannula CA1100c.

There may be the following one-way valves within arrangement 1100A:

one-way valve V1 lAa within separated portion SP11Aa,

one-way valve V1 1 Ab within separated portion SP1 1 Ab ,

one-way valve V1 1 Ac within separated portion SP1 1 Ad, and

one-way valve V1 lAd within separated portion SP11Ae.

Arrangement 1100A comprises the oxygenator device OXY11A which enhances oxygen in the liquid B. Arrangement 1100A is or may be configured to be connected to the oxygenator OXY11A device. Arrangement 1100A is or may be configured such that the outflow of both membrane pump devices MP11Aa, MPllAb or of other pump devices flows through oxygenator device OXY11A. Arrangement 1100A comprises preferably the cannula system CS and is configured to be connected to dual lumen cannula system DL-CS1100 which comprises at least inner cannula CA1100a which is arranged inside of outer cannula CA1100b and to single lumen cannula CA1100c. Arrangement 1100A may be modified if compared with FIG. 11A and may still have the same function.

The function of arrangement 1100A may be as follows:

an arrow Al lAa illustrates inflow from outer cannula CA1100a (RA) through separated portion SP11Ae,

an arrow Al lAb illustrates flow from membrane pump MPllAb to oxygenator device OXY11A via connecting portions CP11Ab and CP11Ad,

an arrow Al lAc illustrates inflow from inner cannula CA1100b (LA) through separated portion

SP1 1 Aa,

an arrow Al lAd illustrates flow from membrane pump MP11Aa to oxygenator device OXY11A via connecting portions CP11Aa, CP1 lAd, and

an arrow Al lAe illustrates flow from output port of oxygenator device OXY11A to single lumen cannula CA1100c.

Thus, all blood Bl, B2 which flows into arrangement 1100A is oxygenated and a pulsatile flow is provided in outflow of arrangement 1100A, see arrows Al lAe and A1100e.

FIG. 11B illustrates a second embodiment of a pump arrangement Arrl1B for the pBiVADR assist system or arrangement 1000, 1100B of FIG. 11 including an oxygenator device OXY11B and allowing pulsatile outflow.

Other parts of the arrangement 1100B which is illustrated in FIG. 11B correspond to parts mentioned in FIG. 11A, e.g.:

separated portions SP12Ba to SP12Bf to separated portions SP11Aa to SP11Af respectively,

connecting portions CP12Ba to Cl2Bc to connecting portions CP11Aa to CP11Ac,

oxygenator device OXY12B to oxygenator device OXY11A,

one-way valves V12Ba to V12Bd to one-way valves V11Aa to V11Ad respectively, and

IABP console IAPB12B to IABP11A.

There may be the following differences:

no connecting portion which corresponds to connecting portion CP1 lAd,

a connecting portion CP11Be downstream of oxygenator device OXY11B,

separated portion SP11Bb bypasses oxygenator device OXY11B and is directly connected to connecting portion CP11Be,

optional one-way valves V11Be within separated portion SP11Bb, e.g. there may be two one-way valves V11Bb and V11Be within this separated portion, and/or V11Bf, within portion P11B2,

a portion P11B2 from an output port of oxygenator device to connecting portion CP11Be, and

a portion P11B3 from connecting portion CP11Be to cannula CA1100c.

Arrangement 1100B comprises oxygenator device OXY11B which enhances oxygen in liquid B. Arrangement 1100B is or may be configured to be connected to oxygenator OXY11B device. Arrangement 1100B is or may be configured such that the outflow of one pump device, for instance of membrane pump device MP11Bb, of the at least two pump devices, for instance membrane pump devices MP11Ba, MP11Bb flows through oxygenator device OXY11A but not the outflow of the other pump device, for instance membrane pump MP11Ba, of the at least two pump devices, for instance of membrane pump devices MP11Ba, MP11Bb. Arrangement 1100B may comprise cannula system CS and may be configured to be connected to dual lumen cannula system (DL-CS1100) which comprises at least one inner cannula CA1100a which is arranged inside of an outer cannula CA1100b and to a single lumen cannula CA1100c. Arrangement 1100B may be modified if compared with FIG. 11B and may still have the same function.

The function of arrangement 1100B is as follows:

an arrow A 11Ba illustrates inflow from outer cannula CA1100a (RA) through separated portion SP11Be,

an arrow A 11Bb illustrates flow from membrane pump MP11Bb to oxygenator device OXY11B via connecting portion CP11Bb,

an arrow A 11Bc illustrates inflow from inner cannula CA1100b (LA) through separated portion SP11Ba,

an arrow A 11Bf illustrates flow from membrane pump MP11Ba to connecting portion CP11Be bypassing oxygenator device OXY11B,

an arrow Al lBg illustrates flow from output port of oxygenator device OXYllA to connecting portion CP11Be, and

an arrow Al lBh illustrates flow from connecting portion CP11Be to single lumen cannula CA1100c.

Thus, only blood B1 which flows into arrangement 1100B from right atrium RA is oxygenated. Blood B2 from left atrium LA which is already oxygenated by lung L is not oxygenated within arrangement 1100B.

Pulsatile flow is provided in outflow of arrangement 1100B, see arrows Al lBh and A1100e.

FIG. 12 illustrates a veno-arterial extracorporeal membrane oxygenation system or arrangement 1200 with drain in right atrium RA and/or superior vena cava SVC and/or inferior vena cava IVC and return cannula CA1200b to aorta AO, e.g. to ascending aorta aAO or to descending aorta dAO. An input cannula CA1200a and cannula CA1200b may be a single lumen cannula.

Other parts of the arrangement 1200 which is illustrated in FIG. 12 correspond to parts mentioned in FIG. 5, e.g.:

separated portions SP12a to SP12f to separated portions SP5a to SP5f respectively,

connecting portions CP12a to CP12c to connecting portions CP5a to CP5c,

oxygenator device OXY12 to oxygenator device OXY5,

one-way valves V12a to V12d to one-way valves V5a to V5d respectively, and

IABP console IAPB12 to IABP5.

There may be the following differences:

cannula CA1200a extends though superior vena cava, through right atrium RA into inferior vena cava IVC.

cannula CA1200a comprises an intermediate portion IP1200a1and an intermediate portion IP1200a2,

intermediate portion IP1200a1 comprises openings OP1200a1, and

intermediate portion IP1200a2 comprises openings OP1200a2.

The function of arrangement 1200 is as follows:

arrows A1200a1, A1200a2 and A1200a3 illustrates an inflow in cannula CA1200a through openings OP1200a1, OP1200a2 and through distal opening D01200a respectively,

an arrow A1200c illustrates a flow through oxygenator device OXY12,

an arrow A1200d illustrates an outflow through separated portion SP12e,

an arrow A1200e illustrates an outflow at distal portion DP1200b1 (variant 1) of cannula CA1200b, and

an arrow A1200f illustrates an alternative outflow at distal portion DP1200b2 (variant 2) of cannula CA1200b.

Again, three way stop cocks may be used to enable change of the membrane pumps MP12a, MP12b during operation of arrangement 1200.

FIG. 13 illustrates a further arrangement comprising two membrane pumps MP13a and MP13b operated in parallel. Five Y-connectors and three one-way valves may be used.

Membrane pump MP13a has only one blood port which is used as inlet port and as an outlet port, e.g. a variable volume reservoir port RP. The single liquid port of membrane pump MP13a is connected to a first connecting portion CP13a, for instance a first Y-connector, via a separated portion SP13c.

First connecting portion CP13a is also connected to a separated portion SP13a which is an inlet portion or inflow portion of arrangement 1300. Furthermore, first connecting portion CP13a is also connected to a separated portion SP13b.

Membrane pump MP13b has only one blood port which is used as inlet port and as an outlet port, e.g. a variable volume reservoir port RP. The single liquid port of membrane pump MP13b is connected to a second connecting portion CP13b, for instance a second Y-connector, via a separated portion SP13f.

Second connecting portion CP13b is also connected to a separated portion SP13d. Furthermore, second connecting portion CP13a is also connected to a separated portion SP13e which is a further inlet portion or inflow portion of arrangement 1300.

There is a third connecting portion CP13c, for instance a third Y-connector. Third connecting portion CP13c is connected with separated portion SP13b and with separated portion SP13d as well as with a separated portion SP13g which forms an outflow portion of arrangement 1300.

A fourth connecting portion CP13d may also be realized using a Y-connector, for instance a fourth Y-connector. Fourth connecting portion CP13d is connected with separated portion SP13a and with separated portion SP13e as well as with a separated portion SP13h which forms an inflow portion of arrangement 1300.

A fifth connecting portion CP13e may also be realized using a Y-connector, for instance a fifth Y-connector. Fifth connecting portion CP13e is connected with separated portion SP13h and with separated portion SP13g as well as with a separated portion SP13i which forms a common inflow portion and outflow portion of arrangement 1300.

There may be the following one-way valves in arrangement 1300:

a one-way valve V13a within first separated portion SP13a,

a one-way valve V13b within second separated portion SP13b,

a one way valve V13c within separated portion SP 13d, and

a one-way valve V13d within separated portion SP4e.

One-way valves V4a to V4c are symbolized by “arrows” which to not necessarily correspond to the internal structure of these valves. However, the direction of the “arrow” corresponds to the flow direction which is possible through the respective valve V13a to V13c.

Membrane pumps MP13a and MP13b are connected to an IABP console IABP13 at their gas inlets. A further connecting portion CP13f connects the air ports of membrane pump MP13a and MP13b to IABP console IABP13.

The function of arrangement 1300 is as follows:

Both membrane pumps MP13a and MP13b suck in blood in an aspiration phase. This blood is sucked through separated portion SP13i and is transferred only to connecting portion CP13d but not to connecting portion CP13c because of the one-way valves V13a to V13d. One-way valves V13a and V13d allowing inflow through separated portions SP13a and SP13e. One-way valves V13b and V13c blocking inflow through separated portions SP13b and SP13c.

Furthermore, during aspiration phase, incoming blood is distributed or branched off at connecting portion CP13d to separated portion SP13a and to separated portion SP13e. Thereafter, blood flows through separated portion SP13a and one-way valve V13a into membrane pump MP13a whereby one-way valve V13a prevents drainage of blood from separated portion SP13b. Likewise, blood flows through separated portion SP13e and one-way valve V13d into membrane pump MP13b whereby one-way valve V13c prevents drainage of blood from separated portion SP13d.

Both membrane pumps MP13a and MP13b expulse blood in an expulsion phase. With regard to membrane pump MP13a, blood is expulsed through separated portion SP13c, SP13b and is transferred via connecting portion CP13a to connecting portion CP13c but not to connecting portion CP13d because of the one-way valve V13a which blocks blood flow in the expulsion phase. With regard to membrane pump MP13b, blood is expulsed through separated portion SP13f, SP13d and is transferred via connecting portion CP13b to connecting portion CP13c but not to connecting portion CP13d because of the one-way valve V13d which blocks blood flow in the expulsion phase.

Furthermore, during the expulsion phase blood is collected at connecting portion CP13 and guided as a common flow through separated portion SP13g, via connecting portion CP13e to separated portion SP13i.

One-way valves V13a and V13d allowing inflow through separated portions SP13a and SP13e. One-way valves V13b and V13c locking inflow through separated portions SP13b and SP13c.

Thereafter, the aspiration phase and the expulsion phase are repeated, preferably cyclic. Thus, for instance a bidirectional cannula CA1300 may be connected to separate portion SP13i.

The technical effect of arrangement 1300 is that unwanted fluid flows between the two membrane pumps MP13a, MP13b are reliably avoided.

More one-way valves may be used than four one-way valves V13a to V13d. However, there may also be reason to omit at least one of the V13a to V13d, for instance if decoupling of both membrane pumps

MP13a, MP13b has not to be completely.

Arrangement 1300 may be used instead of pump arrangements Arr2, Arr3a, Arr6b, Arr7b, Arr9b or Arr10b or for other pump arrangements.

Furthermore, connecting portion CP13e and/or separated portion SP13h and/or separated portion SP13i are optional if a cannula is used which is different from a bidirectional cannula, for instance two single lumen cannulas or a dual lumen cannula. (??RECO??)

In all embodiments using two membrane pumps and an oxygenator device, e.g. arrangements 400 (FIG. 4), 500 (FIG. 5), 800 (FIG. 8), 1100A (FIG. 11A) and 1100B (FIG. 11B) at least one of the membrane pumps or both membrane pumps may be replaced by a parallel connection of two membrane pumps, for instance by a pump arrangement Arr2 or by the arrangement illustrated in FIG. 13. Thus, overall four membrane pumps may be used in these arrangements 400, 500, 800, 1100A and/or 1100B.

In all embodiments, the shorter cannula may be inserted via the right jugular vein, for instance in order to reduce the number of curvatures during insertion. In all cases that mention cannulas in this description, the cannula(s) may have at least one variable diameter arrangement, e.g. a cage arrangements and/or balloon. The variable diameter arrangement may be located around at least one hole/opening of the cannula. At least one membranes may also be used on the cage arrangement(s).

In all embodiments only one pump may be used or two pumps in parallel may be used, preferably at all locations where a single pump is shown, for instance also in FIGS. 4, 5, 8, 11A, 11B and 12, e.g. four pumps may be used, e.g. membrane pump devices. For example, membrane pump device MP12a may be replaced by two pumps in parallel. Alternatively, other pumps than membrane pumps may be used in all embodiments, for instance centrifugal pumps, radial pumps, diagonal pumps, in combination with a variable volume reservoir. Furthermore, these pumps may also be used without a variable volume reservoir.

In all embodiments optional three way stop cocks may be used, for instance 3WSC2a, 3WSC2b, which may allow changing of pumps during operation of the arrangement 200 to 1200.

In all embodiments, optional one-way valves or other valves may be used within or at the distal end of the cannulas in addition to the one-way valves in the separated portions. These further one-way valves prevent that blood flows into outflow openings which are mainly used as outflow openings or that blood flows out of inflow openings which are mainly used as inflow openings. Alternatively and/or additionally, there may be one-way valves or other valves within an intermediated portions of the cannulas which have the same purpose. This is similar to flaps within the veins of the human body which flaps prevent backflow during the systole. These backflow preventing valves may be used in unidirectionally used cannula, especially in a single lumen cannula and/or in a dual lumen cannula as mentioned above.

In other words, the following is proposed. Membrane pumps may be used as drive pumps which was tested. The control unit of the membrane pumps may be an IABP console which does not only drive a 40 ml (milliliter) or 40 cc (cubic centimeter) but also two pumps of for instance 40 ml or of 40 ml plus and/or minus 10 percent.

There are the following variants:

two or more membrane pump devices in parallel, and/or

two or more membrane pump devices in series, and/or

two or more membrane pump devices in series with integrated oxygenator (pulsatile outflow/ECMO may be possible), and/or

two or more membrane pump devices in parallel with integrated oxygenator, this may result in a pulsatile ECMO in a variant.

Furthermore, it is possible to use in all embodiments that are mentioned above an inner surface of the lumen portion that comprises a helically surface structure. The helically surface structure may have the effect that the fluid flow within the cannula is rotated as it moves through the cannula. Turbulences may be reduced thereby and/or it may be possible to reach much higher flow rates compared to cannulas that have a smooth inner surface, i.e. that do not have helical surface structures on their inner surfaces. However, it is of course possible to use cannulas without helical inner surface features, if for instance lower flow rates are necessary. The spirally turned flow and/or the rotated flow may prevent clotting of blood cells if the fluid flow comprises blood, especially in slow flow rate conditions. However, there may also be advantages if the fluid flow does not contain blood. The rotating flow may be a laminar flow.

A variable diameter arrangement may be used at the distal tip of all cannulas mentioned above, e.g. in the FIGS. 1 to 12, for instance a cage arrangement. The cage may comprise metal wires. It is also possible to use another material than a metal, for instance a natural and/or biological material, especially cellulose, for instance cellulose that is treated to increase the hardness. Compatibility with body 100 and/or with blood may be improved thereby. Single end-hole cannulas may be used, preferably in combination with a cage arrangement, which may prevent damage of vessels due to inflow into the body, suction of tissue into the cannula and which may enable a secure holding of the cannula within the body. Furthermore, single end-hole cannulas may also reduce shear stress compared for instance to multi-hole tip cannulas, which may nevertheless also be used.

In all embodiments one of the following methods may be used to bring or guide a guide wire and/or a catheter around or along the acute angle within the left ventricle LV. At least one snare may be used to catch the catheter and/or the guide wire in the left ventricle LV. The methods may be performed independent whether there is jugular access or a femoral access or another access for the catheter and/or the guide wire.

Variant A (catching the catheter with the snare):

1) Introducing a catheter through the right atrium RA, the atrial septum AS (a puncturing step may be performed earlier or using the catheter, e.g. using a needle and/or RF (radio frequency) tip/wire within the catheter). The catheter may be introduced further through the hole (puncture) in the atrial septum AS through left atrium LA, through mitral valve MV into the left ventricle LV.

2) Introducing a snare from descending aorta AO through aortic valve AV into left ventricle LV. This step may be performed also before step 1.

3) Catching the catheter in the left ventricle LV using the snare.

4) Pulling the snare and the distal end of the catheter therewith to the aorta AO.

5) Introducing a guide wire through the catheter.

6) Forwarding the guide wire out of the distal end of the catheter. Slight loosening of the snare may be optionally performed thereby.

7) As the guide wire is already within the snare, pull back the snare to a region in which only the guide wire is located but not the catheter.

8) Fix the guide wire using the snare, e.g. contract the snare and/or tighten the snare.

9) Optional, externalizing for instance the distal end of the guide wire out of the body. This step is optionally, because the proximal end of the snare is already outside of the body.

10) Remove catheter, e.g. pull back the catheter.

11) Introduce cannula using the guide wire, e.g. pushing the cannula along and/or over the guide wire until it is on its final place.

Variant B (catching the guide wire with the snare):

1) Introducing a catheter through the right atrium RA, through the atrial septum AS (a puncturing step may be performed earlier or through catheter, use needle and/or RF (radio frequency) tip/wire). Introducing the catheter further through left atrium LA, mitral valve MV into the left ventricle LV.

2) Introducing a guide wire through the catheter until the distal end of the guide wire comes out of the distal end of the catheter within the left ventricle LV. The RF wire may be used also as a guide wire.

3) Introducing a snare from descending aorta AO through aortic valve AV into left ventricle LV. This step may be performed before step 1 and/or before step 2.

3) Catching the distal end of the guide wire in the left ventricle LV using the snare.

4) Fixation of the guide wire using the snare.

5) Pulling the snare and the distal end of the guide wire therewith to the aorta AO.

6) Optional, externalizing guide wire by pulling it out of the body using the snare. This step is optional as the snare is already outside of the body from where it has been introduced.

7) Remove catheter, e.g. by pulling it back along the guide wire.

8) Introduce cannula over/along the guide wire until it is on place.

The following method may also be used in all corresponding embodiments for introducing a cannula jugularly transseptally:

1) Introduce a first snare into an internal jugular vein IJV, for instance into the right jugular vein RJV or into the left jugular vein LJV.

2) Advancing the first snare to inferior vena cava IVC.

3) Introducing a catheter into a common femoral vein CFV (left or right).

4) Advancing the catheter through the first snare into an inferior vena cava IVC.

5) Advancing the catheter through the first snare into the vena cava VC in an antegrade fashion.

6) Advancing the catheter through the first snare into the right atrium RA in an antegrade fashion.

7) Advancing the catheter through the first snare and from the right atrium RA transseptally through the atrial septum into the left atrium LA in an antegrade fashion. Puncturing of atrial septum may have been performed earlier. Alternatively, the catheter is used to puncture the atrial septum, for instance using a needle or using a RF (radio frequency) wire/tip which is introduced trough the catheter.

8) Advancing the catheter through the first snare and advancing the catheter across the mitral valve MV and into the left ventricular outflow tract, e.g. the left ventricle LV.

9) Advancing a second snare in the ascending aorta AO catching and snaring a distal portion of the catheter (Variant A) within the left ventricle LV. The second snare may optionally be introduced through an artery, which may include, but is not limited to, a radial artery, a brachial artery, an axillary artery, a subclavian artery, a carotid artery, or common femoral artery, and advanced retrograde into the aorta AO and into the left ventricle LV. The second snare may be already introduced before the catheter is introduced.

Alternatively, a guide wire may be inserted into the catheter until a distal end of the guide wire comes out of a distal opening of the catheter. This distal end of the guide wire is then caught and snared within the left ventricle (Variant B)

10) Pulling the catheter (Variant A) or the guide wire (Variant B) into the aorta AO in an antegrade fashion using the second snare.

11) In variant A, advancing a guide wire through the catheter and through the first snare in antegrade fashion to the ascending aorta AO and through the second snare. Snaring the distal end of the guide wire in variant A but not the catheter.

12) In both variants A and B remove the catheter with the guide wire remaining in the heart H and through the first snare after the catheter is removed.

13) Externalizing a proximal portion of the guide wire from femoral vein, through inferior vena cava IVC, through inferior vena cava SVC, into the internal jugular vein IJV and then out of the internal jugular vein IJV using the first snare, for instance left jugular vein LJV or right jugular vein RJV. In some embodiments the snare may externalize a different portion of the guide wire, for instance an intermediate portion.

14) Advancing a cannula using the guide wire and/or along and/or over the guide wire from the internal jugular vein IJV. The cannula may be any of the cannulas described in this specification or known in the art. Especially, an outer cannula may be advanced over the guide wire from the internal jugular vein IJV. An inner cannula may optionally be advanced through a port proximal of the distal end of the outer cannula. The inner cannula and the outer cannula may be positioned as described in this description, or if a single multi-lumen cannula is used, it may be positioned in a similar manner.

15) Optionally, a distal portion of the guide wire may be externalized out of the body through the artery. This step is optional because the second snare is already externalized and may form a secure anchor for the distal portion of the guide wire.

Subclavian arteries/veins or other arteries/veins may be used for introducing the snare(s) because the snares require smaller diameters, e.g. less than 10 French (1 French equal to 1/3 mm (millimeter)) or less than 8 French, e.g. more than 3 French, compared to the diameters of the cannula(s).

In the following details of a method for puncturing transseptally through the atrial septum AS of the heart H are provided. However, other methods may be used as well, for instance using a needle.

A catheter and/or a wire may be used which has a distal tip which can be heated, for instance using RF (radio frequency) energy, alternating current (ac), direct current (dc) etc. Thus, e.g. a hole may be burned into the septum, e.g. the atrial septum AS, during puncturing, for instance using temperatures above 100° C. (degrees Celsius) or above 200° C., less than 1000° C. for instance.

The RF (radio frequency) may be in the range of 100 kHz (kilohertz) to 1 MHz (Megahertz) or in the range of 300 kHz to 600 kHz, for instance around 500 MHz, i.e. in the range of 450 kHz to 550 kHz, e.g. 468 kHz.

The power of the radio frequency energy may have a maximum of 50 Watt. A power range of 5 W (watt) to 100 W may be used, for instance a range of 10 W to 50 W.

A sinus current/voltage may be used for the RF. The sinus current/voltage may be continuous. Alternatively, a pulsed sinus current/voltage may be used for the RF.

All parameters or some of the parameters of the RF equipment may be adjustable by an operator who performs the puncturing, for instance dependent on the specifics of the septum, e.g. normal septum, fibrotic septum, aneurysmal septum, etc. Preferably, the power may be adjustable.

A solution of Baylis Medical (may be a trademark), Montreal, Canada may be used, for instance NRG® trans-septal needle or Supra Cross® RF Wire technology. RF generator of type RFP-100A or a further development of this model may be used. This RF generator uses for example a frequency of 468 kHz (kilohertz).

A single puncture of the septum may be performed from a jugular access or from a femoral access or from another appropriate access using the RF energy. Smaller angles may be possible for the catheter if for instance compared with a needle.

Alternatively, the RF method may be used also if two separate punctures are made in the septum. However, usage of needles is possible as well. One of the punctures using the RF method may be made through left jugular vein LJV and the other puncture of the atrial septum AS may be made through the right jugular vein RJV.

It is possible to introduce both guide wires first through the atrial septum AS. Preferably, separate holes are used for each of the guide wires. Guide wire(s) may be used which include an RF tip. Alternatively, the wire(s) having the RF tip may be pulled back and a further wire may be introduced through the catheter.

Only after both guide wires are in place, both cannulas may be introduced using a respective one of the guide wires.

Alternatively, the first puncture may be performed using RF energy or a needle. Thereafter, the first cannula for blood transfer is inserted using the first guide wire. After insertion of the first cannula, the second puncture may be made. A second guide wire or the first guide wire may be used to introduce the second cannula.

Puncturing of the atrial septum may be assisted by at least one medical imaging method, preferably by at least two medical imaging methods.

US (ultra-sonic) echo imaging may be used to visualize the movement of heart H and the location of the valves of heart H. No dangerous radiation may result from ultra-sonic imaging. An ultra-sonic transmitter may be introduced for instance via the esophagus, e.g. trans esophagus echo (TEE) may be used.

X-ray radiation preferably in combination with fluorescence (fluoroscopy), may be used in order to visualize the location of catheters (comprising for instance at least one X-ray marker, or the devises are usually radiopaque) and/or the location of guide wire(s), snares etc.

Thus, transseptal puncturing or puncturing of other tissue may be guided by TEE and by fluoroscopy or by other imaging methods. At least two different image generating methods may be used.

In all embodiments mentioned above, it is also possible to use a soft guide wire and a stiffer guide wire which does not bend so easy if compared with the soft guide wire. The following steps may be performed, preferably in combination with snaring:

1) Introduce a soft guide wire.

2) Introduce catheter using the soft wire as a guide.

3) Optionally, remove soft wire, for instance by pulling back the soft wire out of the catheter.

4) Introduce stiffer guide wire into the catheter, e.g. there may be a change of wire from soft wire to the stiffer wire.

The catheter may be removed, e.g. pulled back. Thereafter, the stiffer wire may be used to introduce a cannula or cannulas.

Although embodiments of the present disclosure and their advantages have been described in detail, it should be understood that various changes, substitutions and alterations may be made therein without departing from the spirit and scope of the disclosure as defined by the appended claims. For example, it will be readily understood by those skilled in the art that many of the features, functions, processes and methods described herein may be varied while remaining within the scope of the present disclosure.

Moreover, the scope of the present application is not intended to be limited to the particular embodiments of the system, process, manufacture, method or steps described in the present disclosure. As one of ordinary skill in the art will readily appreciate from the disclosure of the present disclosure systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps.

The embodiments mentioned in the first part of the description may be combined with each other. The embodiments of the description of Figures may also be combined with each other. Further, it is possible to combine embodiments mentioned in the first part of the description with examples of the second part of the description which relates to FIGS. 1 to 13.

1. Arrangement (200 to 1200) for transporting a liquid (B) through a cannula system (CS), comprising:
a liquid guiding system (LGS) comprising at least three separated portions (SP2a, SP2b, SP3c) which define separate liquid guiding portions of the liquid guiding system (LGS), and a connecting portion (CP2 to CP12) which fluidically connects the at least three separated portions (SP2a, SP2b, SP3c) and which comprises a lumen that branches out into at least two lumens,
wherein the liquid guiding system (LGS) is configured to be connected to a pump arrangement (Arr2 to Arr12) which drives a flow of the liquid (B), and
wherein the liquid guiding system (LGS) is configured to be connected to a cannula system (CS) which is adapted to be inserted into a body of a human or of an animal and which comprises an inflow opening and an outflow opening of the liquid guiding system (LGS).

2. Arrangement (200, 300, 600, 700, 900, 1000) according to claim 1, wherein a first separated portion (SP2a, SP3a, SP9d) of the at least three separated portions (SP2a, SP2b, SP2c; SP3a, SP3b, SP3c; SP9d, SP9e, SP9f) and a second separated portion (SP2b, SP3b, SP9e) of the at least three separated portions (SP2a, SP2b, SP2c; SP3a, SP3b, SP3c; SP9d, SP9e, SP9f) are configured to be connected to a pump arrangement (Arr2, Arr3) which drives a flow of the liquid (B) through the liquid guiding system (LGS), wherein the first separated portion (SP2a, SP3a, SP9d) is different from the second separated portion (Sp2b, SP3b, SP9e),
wherein the arrangement (200, 300, 600, 700, 900, 1000) is configured such that a third separated portion (SP2c, SP3c, SP9f) of the at least three separated portions (SP2a, SP2b, SP2c; SP3a, SP3b, SP3c; SP9d, SP9e, SP9f) is a common inlet and outlet portion through which flow flows coming from the first separated portion (SP2a, SP3a, SP9d) and from the second separated portion (SP2b, SP3b, SP9e) and/or through which third separated portion (SP2c, SP3c, SP9f) flow flows which then flows to the first separated portion (SP2a, SP3a, SP9d) and to the second separated portion (SP2b, SP3b, SP9e).

3. Arrangement (200, 300) according to claim 2, preferably comprising the pump arrangement (Arr2, Arr3),
wherein the pump arrangement (Arr2, Arr3) comprises at least two pump devices (MP2a, MP2b; MP3a, MP3b),
wherein a first pump device (MP2a, MP3a) of the at least two pump devices (MP2a, MP3a) is configured to be connected to the first separated portion (SP2a, SP3a), and
wherein a second pump device (MP2b, MP3b) of the at least two pump devices (MP2a, MP2b; MP3a, MP3b) is configured to be connected to the second separated portion (Sp2b, SP3b).

4. Arrangement (200, 300, 900) according to claim 2 or 3, wherein each of the at least three separated portions (SP2a, SP2b, SP2c; SP3a, SP3, SP3c; SP9d, SP9e, SP9f) is configured to allow a bidirectional flow of the liquid (B).

5. Arrangement (200, 300, 900) according to any one of the claims 2 to 4, comprising the cannula system (CS), wherein the cannula system comprises:
two single lumen cannulas (CA500a, CA500b; CA600a, CA600b; CA800a, CA800b; CA1000a, CA1000b; CA1200a, CA1200b), preferably configured for right ventricle (RV) assist (pRVAD) or for left ventricle (LV) assist (pLVAD®) or for bi ventricle assist (pBiVAD®) or for pulmonary artery (PA) to left atrium (LA) or left ventricle (LV) blood transport or for veno-arterial extracorporeal membrane oxygenation, or
a dual lumen cannula system (DL-CS700, DL-CS1100) comprising at least one inner cannula (CA700a) which is inserted into an outer cannula (CA700b), preferably configured for left ventricle (LV) assist (pLVAD®) or for biventricular assist (pBiVAD®),
a bidirectional cannula (CA200, CA300a, CA900a) which is configured to be connected to the third separated portion (SP2c, SP3c, SP9f), preferably configured for renal assist or for right ventricle (RV) assist (pRVAD) or for pulmonary artery (PA) to left atrium (LA) or left ventricle (LV) blood (B) transport.

6. Arrangement (400 to 1200) according to claim 1, wherein a first separated portion (SP4a) of the at least three separated portions (SP4a, SP4b, SP4c) is an inflow portion of the liquid guiding system (LGS),
wherein a second separated portion (SP4b) of the at least three separated portions (SP4a, SP4b, SP4c) is an outflow portion of the liquid guiding system (LGS),
wherein the first separated portion (SP4a) is different from the second separated portion (SP4b),
wherein a third separated portion (SP4c) of the at least three separated portions (SP4a, SP4b, SP4c) is configured to be connected to a pump arrangement (Arr4) which drives a flow of the liquid (B), and
wherein the arrangement (400 to 1200) is configured such that the third separated portion (SP4c) is a portion through which flow coming from the first separated portion (SP4a) flows and/or through which flow flows which flows then to the second separated portion (SP4b).

7. Arrangement (400 to 1200) according to claim 6, wherein the first separated portion (SP4a) but not the second separated portion (SP4b) or the second separated portion (SP4b) but not the first separated portion (SP4a) comprises a one-way valve (V4a, V4b) or wherein alternatively the first separated portion (SP4a) comprises a first one-way valve (V4a) and the second separated portion (SP4b) comprises a second one way valve (V4b).

8. Arrangement (600, 700, 900, 1000) according to claim 6 or 7, preferably comprising the pump arrangement (Arr6b, Arr7a, Arr9a, Arr10a),
wherein the pump arrangement (Arr6b, Arr7a, Arr9a, Arr10a) comprises only one pump device (MP6, MP7, MP9, MP10), and
wherein the pump device (MP6, MP7, MP9, MP10) is configured to be connected to the third separated portion (SP6c, SP7c, SP9c, SP10c).

9. Arrangement (600, 700, 900, 1000) according to claim 8, wherein the arrangement (600, 700, 900, 1000) comprises the cannula system (CS), and wherein the cannula system (CS) comprises:
two single lumen cannulas (CA600a, CA600b; CA1000a, CA100b) defining an inflow opening and an outflow opening of the liquid guiding system (LGS), preferably configured for left ventricle (LV) assist (pLVAD®) or for pulmonary artery (PA) to left atrium (LA) or left ventricle (LV) blood transport, or a dual lumen cannula system (CS700) which comprises at least one inner cannula (CA700b) which is arranged inside of an outer cannula (CA700a), preferably configured for left ventricle (LV) assist (pLVAD), or
a bidirectional cannula (CA900) defining an inflow opening and an outflow opening of the liquid guiding system (LGS) and comprising at least one internal valve or internal valve function, preferably configured for pulmonary artery (PA) to left atrium (LA) or left ventricle (LV) blood transport.

10. Arrangement (900, 1000) according to claim 8 or 9, wherein the arrangement (900, 1000) comprises a device (D9, D10) for carbon dioxide removal from the liquid (B), and
wherein the arrangement (900, 1000) is configured to be connected with the device (D9, D10) for carbon dioxide removal but not with an oxygenator device which enhances oxygen in the liquid.

11. Arrangement (400 to 1200, 1300) according to claim 6 or 7, preferably comprising the pump arrangement (Arr4 to Arr12),
wherein the pump arrangement (Arr4 to Arr12) comprises at least two pump devices (MP4a, MP4b, MP5a, MP5b; MP6a, MP6b; MP7a, MP7b; MP8a, MP8b; MP9a, MP9b; MP10a, MP10b; MP11Aa, MP11Ab; MP12a, MP12b).

12. Arrangement (600, 700, 900, 1000, 1100A, 1100B, 1300) according to claim 11, wherein both pump devices (MP6a, MP6b; MP7a, MP7b; MP9a, MP9b; MP10a, MP10b, MP11Aa, MP11Ab; MP11Ba, MP11Bb) are configured to be fluidically connected in parallel.

13. Arrangement (600, 700, 900, 1000, 1100A, 1100B, 1300) according to claim 12, wherein the arrangement (600, 700, 900, 1000, 1100A, 1100B) comprises the cannula system (CS) and wherein the cannula system (CS) comprises:
two single lumen cannulas (CA600a, CA600b; CA1000a, CA100b) defining an inflow opening and an outflow opening of the liquid guiding system (LGS), preferably configured for left ventricle (LV) assist (pLVAD®) or for pulmonary artery (PA) to left atrium (LA) or left ventricle (LV) blood (B) transport, or a dual lumen cannula system (DL-CS700, DL-CS1100) which comprises at least one inner cannula (CA700b, CA1100a) which is arranged inside of an outer cannula (CA700a, CA1100b), preferably configured for left ventricle (LV) assist (pLVAD®) or for bi-ventricle assist (pBiVAD®), or
wherein the arrangement (900, 1300) is configured to be connected to a bidirectional cannula (CA900, 1300) defining an inflow opening and an outflow opening of the liquid guiding system (LGS) and comprising at least one internal valve or internal valve function, preferably configured for pulmonary artery (PA) to left atrium (LA) or left ventricle (LV) blood (B) transport.

14. Arrangement (1100A, 1100B) according to claim 12 or 13,
wherein the arrangement (1100A, 1100B) is configured to be connected to an oxygenator device (OXY11A, OXY11B) which enhances oxygen in the liquid (B).

15. Arrangement (1100A) according to claim 12, wherein the arrangement (1100A) comprises an oxygenator device (OXY11A) which enhances oxygen in the liquid (B),
wherein the arrangement (1100A) is configured to be connected to the oxygenator device (OXY11A),
wherein the arrangement (1100A) is configured such that the outflow of both pump devices (MP11Aa and MP11Ab) flows through the oxygenator device (OXY11A),
wherein preferably the arrangement (1100A) comprises the cannula system (CS) and is configured to be connected to a dual lumen cannula system (DL-CS1100) which comprises at least one inner cannula (CA1100a) which is arranged inside of an outer cannula (CA1100b) and to a single lumen cannula (CA1100c).

16. Arrangement (1100B) according to claim 12, wherein the arrangement (1100B) comprises an oxygenator device (OXY11B) which enhances oxygen in the liquid,
wherein the arrangement (1100B) is configured to be connected to the oxygenator (OXY11B) device, and
wherein the arrangement (1100B) is configured such that the outflow of one pump device (MP11Bb) of the at least two pump devices (MP11Ba, MP11Bb) flows through the oxygenator device (OXY11A) but not the outflow of the other pump device (MP11Ba) of the at least two pump devices (MP11Ba, MP11Bb),
wherein preferably the arrangement (1100B) comprises the cannula system (CS) and is configured to be connected to a dual lumen cannula system (DL-CS1100) which comprises at least one inner cannula (CA1100a) which is arranged inside of an outer cannula (CA1100b) and to a single lumen cannula (CA1100c).

17. Arrangement (400, 500, 800, 1200) according to claim 11, wherein the at least two pump devices (MP4a, MP4b, MP5a, MP5b; MP8a, MP8b; MP12a, MP12b) are configured to be connected fluidically in series.

18. Arrangement (400, 500, 800, 1200) according to claim 17, wherein the arrangement (400, 500, 800, 1200) comprises the cannula system (CS) and wherein the cannula system (CS) comprises:
two single lumen cannulas (CA500a, CA500b; CA800a, CA800b; CA1200a, CA1200b) defining an inflow opening and an outflow opening of the liquid guiding system (LGS), preferably configured for right ventricle (RV) assist (pRVAD®) or for biventricular assist (pBiVAD®) or for veno-arterial extracorporeal membrane oxygenation (V-A ECMO), or
a dual lumen cannula system which comprises at least one inner cannula which is arranged inside of an outer cannula, or
a bidirectional cannula (CA300a) which comprises an inflow opening (DO300a) and an outflow opening (IO300a) of the liquid guiding system (LGS) and comprising at least one internal valve or internal valve function, preferably configured for right ventricle (RV) assist (pRVAD®).

19. Arrangement (400, 500, 800, 1200) according to claim 17 or 18, wherein the arrangement (400, 500, 800, 1200) may comprise an oxygenator device (OXY4, OXYS, OXY8, OX12) which enhances oxygen in the liquid, and
wherein the arrangement (400, 500, 800, 1200) is configured to be connected to the oxygenator device (OXY4, OXYS, OXY8, OX12).

20. Arrangement (400, 500, 800, 1200) according to claim 19,
wherein the arrangement (400, 500, 800, 1200) is configured such that the outflow of only one pump device (MP4a, MP5a, MP8a, MP12a) of the at least two pump devices (MP4a, MP4b; MP5a, MP5b; MP8a, MP8b; MP12a, MP12b) flows through the oxygenator device (OXY4, OXYS, OXY8, OX12) and that the outflow of the oxygenator device (OXY4, OXYS, OXY8, OX12) flows through the other pump device (MP4b, MP5b, MP8b, MP12b) of the at least two pump devices (MP4a, MP4b; MP5a, MP5b; MP8a, MP8b; MP12a, MP12b).

21. Arrangement (100 to 1200) according to any one of the preceding claims, comprising the pump arrangement (Arr2 to Arr12),
wherein the pump arrangement (Arr2 to Arr12) comprises at least one pump device (MP1 to MP12b) for driving the liquid through the liquid guiding system (LGS),
wherein the arrangement (100 to 1200) is configured to be connected or is connected to the at least one pump device (MP2 to MP12b),
wherein the at least one pump device (MP2 to MP12b) comprises one port through which the liquid is transported in two opposite directions.

22. Arrangement (200 to 1200) according to claim 21, wherein the at least one port (RP) is the inlet and the outlet of a variable volume reservoir.

23. Arrangement (200 to 1200) according to claim 21 or 22, wherein the at least one pump device is a membrane pump (MP2 to MP12b) which comprises at least one flexible membrane (M),
wherein preferably the membrane (M) separates a case (C) of the membrane pump (MP2 to MP12b) in a variable volume reservoir chamber (Ch1) and in a compensation chamber (Ch2).

24. Arrangement (200 to 1200) according to claim 23, wherein the arrangement (200 to 1200) is configured such that the membrane pump (MP2 to MP12b) is driven by an intra-aortic balloon pump device (IABP1 to IABP12) or such that at least two membrane pump devices (MP2 to MP12b) of the pump arrangement (Arr2 to Arr12) are driven by the same intra-aortic balloon pump console or device (IABP1 to IABP12),
wherein preferably a three-port connector is used to distribute a gaseous fluid coming from the intra-aortic balloon pump console or device (IABP1 to IABP12) to the at least two membrane pump devices (MP2 to MP12b) of the pump arrangement (Arr2 to Arr12), preferably depending on electrode signals of a heart (H) within the body.

25. Arrangement (200 to 1200) according to any one of the preceding claims, comprising at least two three way stop cocks (3WSC2a to 3WSC11Bc) which are configured to enable changing and/or removal and/or stopping of at least one pump device (MP2a to MP12b) of the pump arrangement (Arr2 to Arr12) during continuous operation of the arrangement (200 to 1200) by at least one other pump device (MP2a to MP12b) of the pump arrangement (Arr2 to Arr12).

26. Arrangement (200 to 1200) according to any one of the preceding claims, especially according to any one of claim 5, 9, 13 or 18, wherein the cannula system (CS) comprises a single lumen cannula which is a unidirectional cannula and which comprises at least one backflow prevention valve, preferably a one-way valve,
or wherein the cannula system comprises a dual lumen cannula system comprising an outer cannula and an inner cannula which is inserted into the outer cannula, wherein the inner cannula and/or the outer cannula is a unidirectional cannula comprising at least one backflow prevention valve, preferably a one-way valve,
wherein preferably the backflow prevention valve is arranged at or within a distal portion of the cannula.

27. Arrangement (1300) according to any one of the claims 11 to 13, wherein the arrangement (1300) comprises at least four or at least five multi-port element each comprising at least three ports and at least four one-way valves (V13a to V13d),
wherein preferably a first multi-port element comprises a common port which is connected with a single fluid port of a first membrane pump (MP13a), an inlet port and an outlet port,
a second multi-port element comprises a common port which is connected with a single fluid port of a second membrane pump (MP13b), an inlet port and an outlet port,
a third multi-port element comprises a first inlet port, a second inlet port and a common outlet port, wherein the first inlet port of the third multi-port element is connected to the outlet port of the first multi-port element, and wherein the second inlet port of the third multi-port element is connected to the outlet port of the second multi-port element,
a fourth multi-port element comprises a common inlet port, a first outlet port and a second outlet port, wherein the first outlet port of the fourth multi-port element is connected to the inlet port of the first multi-port element, and wherein the second outlet port of the fourth multi-port element is connected to the inlet port of the second multi-port element, and
preferably a fifth multi-port element comprises a common inlet port and outlet port, an outlet port and an inlet port, wherein the outlet port of the fifth multi-port element is connected to the common inlet port of the fourth multi-port element, and
wherein the inlet port of the fifth multi-port element is connected to the common outlet port of the third multi-port element.

28. Arrangement (200 to 1300) according to any one of the preceding claims, comprising:
a variable volume reservoir comprising:
a case (C), a membrane (M) within the case (C), wherein the membrane (M) separates a reservoir chamber (Ch1) within the case (C) from a compensation chamber (Ch2) within the case (C), and a reservoir port (RP) that is connected to the reservoir chamber (Ch1),
a multi-port element which comprises the at least three separated portions (SP4a, Sp4b, SP4c) and a connecting portion (CP4a),
wherein the connecting portion (CP4a) comprises a lumen that branches out into at least two lumens, an input flow connection which is configured to be fluidically connected with a first separated portion (SP4a) of the at least three separated portions (SP4a, Sp4b, SP4c),
an output flow connection which is configured to be fluidically connected with a second separated portion (SP4b) of the at least three separated portions (SP4a, Sp4b, SP4c),
wherein the reservoir port (RP) is configured to be fluidically connected with a third portion (SP4c) of the at least three separated portions (SP4a, Sp4b, SP4c),
an input one-way valve (Va4) within the first separated portion (SP4a) or within the input flow connection,
wherein the input one-way valve (Va4) allows flow in an input flow direction which is directed from the first separated portion (SP4a) to the third separated portion (SP4c) and which blocks flow in the opposite direction, and/or
an output one-way valve (Vb4) within the second separated portion (SP4b) or within the output flow connection,
wherein the output one-way valve (Vb4) allows flow in an output flow direction which is directed from the third separated portion (SP4c) to the second separated portion (SP4b) and which blocks flow in the opposite direction.

29. Kit, comprising the elements of the arrangement (200 to 1200) according to any one of the claims 1 to 28.

30. Method of using the arrangement (200 to 1200) according to any one of the claims 1 to 28 or the kit according to claim 29,
wherein at least one intra-aortic balloon pump device (IABP2 to IABP12) is used to drive the liquid flow (B),
and wherein preferably at least two pump devices (MP2a to MP12b) of the pump arrangement (Arr2 to Arr12) are driven by the same the same intra-aortic balloon pump device (IABP2 to IABP12).

31. Method of using the arrangement (100 to 1200) according to any one of the claims 1 to 28 or the kit according to claim 29 for any one of the following medical applications:
renal support (200), preferably using a bidirectional cannula (CA200),
right ventricle (RV) assist (300 to 500) (pRVAD®), preferably using a bidirectional cannula (CA300a) which is inserted into an outer cannula (CA300b) or two single lumen cannulas (CA500a, CA500b), left ventricle (LV) assist (600, 700) (pLVAD®), preferably using two single lumen cannulas (CA600a, CA600b) or a dual lumen cannula system (DL-CS700),
bi-ventricle assist (800, 1100) (pBiVAD®), preferably using two single lumen cannulas (CA800a, CA800b) or alternatively a dual lumen cannula system (DL-CS1100) and a single lumen cannula (CA1100c) with extra corporeal membrane oxygenation or without extra corporeal membrane oxygenation,
pulmonary artery (PA) drain to left atrium (LA) or to left ventricle (LV) or to aorta (AO), preferably with carbon dioxide removal (900, 1000), preferably using a bidirectional cannula (CA900a) which is inserted into an outer cannula (CA900b) or using two single lumen cannulas (CA1000a, CA1000b),
veno-arterial (VA) extra corporeal membrane oxygenation (1200)(V-A ECMO), preferably using two single lumen cannulas (CA1200a, CA1200b).

32. Method according to claim 31, wherein biventricular assist (800) is realized using a single lumen cannula (CA800a) which is configured to drain blood (B) from the left atrium (LA) through at least one first opening (DO800a) and to drain blood (B) from the right atrium (RA) through at least one second opening (0P800), or
wherein a biventricular assist (1100) is realized using a dual lumen cannula system (DL-CS1100) which is configured to drain blood (B) from the left atrium (LA) through at least one first opening (D01100a) of a first cannula (CA1100a) of the dual lumen cannula system (CS1100A, CS1100B) and to drain blood from the right atrium (RA) through at least one second opening (0P1100) of a second cannula (CA1100b) of the dual lumen cannula system (DL-CS1100),
wherein preferably the first cannula (CA1100a) is inserted into the second cannula (CA1100b) within the body.
